(19) United States
(12) Patent Application Publication
(10) Pub. No.: US 2021/0346665 A1
(43) Pub. Date: Nov. 11, 2021

(54) CONDUCTIVE MICRONEEDLE PATCH FOR ACTIVE AGENT DELIVERY

(71) Applicants: Singapore Health Services Pte Ltd, Singapore (SG); Nanyang Technological University, Singapore (SG)

(72) Inventors: Chenjie XU, Singapore (SG); Bee Tin Goh, Singapore (SG); Zayim Razino D/O Seeni Syed, Singapore (SG); Yuchun Liu, Singapore (SG); Wan Ting Sharon Chew, Singapore (SG)

(21) Appl. No.: 17/278,223

(22) PCT Filed: Sep. 20, 2019
(86) PCT No.: PCT/SG2019/050480

§ 371 Date: Mar. 19, 2021

(30) Foreign Application Priority Data
Sep. 20, 2018 (SG) 10201808197Y
Sep. 13, 2019 (SG) 10201908526Q

(51) Int. Cl. A61M 37/00; A61N 1/04; A61N 1/30; A61K 9/00; A61K 45/06

(52) U.S. Cl. CPC A61M 37/0015; A61N 1/044; A61N 1/306; A61K 9/0021; A61K 45/06; A61M 2210/04; A61M 2037/0053; A61M 2037/0061; A61M 2202/0241; A61M 2210/02; A61M 2037/0023

(57) ABSTRACT

The present disclosure provides for a microneedle array. The microneedle array is useable for delivering an active agent to a subject. The microneedle array includes a base having microneedles disposed thereon, wherein each of the microneedles is formed of (i) a swellable and water-insoluble matrix comprising a crosslinked polymer or (ii) a water-soluble matrix comprising a water-soluble polymer; and a conductive polymer incorporated in the swellable and water-insoluble matrix or the water-soluble matrix. A device configured to deliver an active agent and a method of delivering an active agent through the device, wherein the device includes the microneedle array, are provided herein. A method of producing the microneedle array is also disclosed herein.

### CROSS-REFERENCE TO RELATED APPLICATION

This application claims the benefit of priority of Singapore Patent Application No. 10201808197Y filed 20 Sep. 2018 and Singapore Patent Application No. 10201908526Q filed 13 Sep. 2019, the content of it being hereby incorporated by reference in its entirety for all purposes.

### TECHNICAL FIELD

The present disclosure relates to a microneedle array and method of producing the microneedle array. The microneedles of the microneedle array are at least electrically conductive. The present disclosure also relates to a device and a method to deliver an active agent, which involve the microneedle array.

### BACKGROUND

Ever since local anaesthesia has been introduced, it has been adopted for various medical practice, e.g. dental practice. Local anaesthesia reduces pain and anxiety to allow a variety of medical procedures, e.g. dental procedures, by blocking conduction in the peripheral nerves or inhibiting excitation of nerve endings. Conventionally, local anaesthesia is administered by invasive and painful needle injection, which tend to render fear and phobia in patients, e.g. paediatric dental patients. To relieve discomfort in the case of dental practice, topical anaesthetics that may be applied physically on the surrounding gingiva of the tooth to numb the surface before needle injection has been developed.

Other developments include computerized injections that allow for slow rate anaesthetic delivery to ease discomfort level by controlling flow of the anaesthetic drug. This, however, is a lengthy and time consuming technique that barely eliminates pain and fear associated with hypodermic needle injections. Needle-free devices such as jet injectors force drugs into target tissues through the use of high pressure. Studies, however, showed that fear and poor patient compliance remains during administration due to a stinging sensation and bad after-taste, including more bleeding than traditional injection.

Even with these developments, the anaesthetic drug diffusion process may still necessitate a wait-time of about 4-10 minutes before numbness kicks in.

As an alternative to hypodermic needle injections, transdermal drug delivery (TDD) has been developed to overcome the disadvantages mentioned above. TDD technologies may include electroporation, cavitational ultrasound, microneedles, etc. Of growing interest is the field of microneedles for minimally invasive delivery of drug molecules through skin. Microneedles may penetrate the skin's barrier (i.e. stratum corneum) to create micropores in skin, thereby allowing easy permeation of drug molecules. This method may omit pain and discomfort associated with needle pricking and possibly led to various types of TDD platforms.

Despite the above, conventional microneedle platforms may have limited synergistic effects when used in combination with other delivery platforms to enhance drug delivery, which presents challenges for fast release and/or quick diffusion of drugs from the microneedles into the deep nerves while being minimally invasive.

There is thus a need to provide for a solution that ameliorates one or more of the limitations mentioned for microneedles, even when combined with other delivery platforms. The microneedles should at least be usable in combination with iontophoresis to deliver an active agent at an improved diffusion rate.

### SUMMARY

In a first aspect, there is provided for a microneedle array comprising:

a base having microneedles disposed thereon, wherein each of the microneedles is formed of (i) a swellable and water-insoluble matrix comprising a crosslinked polymer or (ii) a water-soluble matrix comprising a water-soluble polymer; and

a conductive polymer incorporated in the swellable and water-insoluble matrix or the water-soluble matrix.

In another aspect, there is provided for a device configured to deliver an active agent, the device comprising:

a microneedle array, wherein the microneedle array comprises:

a base having microneedles disposed thereon, wherein each of the microneedles is formed of (i) a swellable and water-insoluble matrix comprising a crosslinked polymer or (ii) a water-soluble matrix comprising a water-soluble polymer; and

a conductive polymer incorporated in the swellable and water-insoluble matrix or the water-soluble matrix; and

an iontophoresis unit comprising an anode and a cathode connectable to the microneedle array, wherein the iontophoresis unit is operable to deliver the active agent from the microneedle array.

In another aspect, there is provided for a method of producing a microneedle array, wherein the microneedle array comprises:

a base having microneedles disposed thereon, wherein each of the microneedles is formed of (i) a swellable and water-insoluble matrix comprising a crosslinked polymer or (ii) a water-soluble matrix comprising a water-soluble polymer; and

a conductive polymer incorporated in the swellable and water-insoluble matrix or the water-soluble matrix;

wherein the method comprises:

providing an aqueous solution in a mold, wherein the aqueous solution comprises (i) a functionalized polymer, the conductive polymer and a photoinitiator, or (ii) the water-soluble polymer and the conductive polymer;

irradiating the aqueous solution to form the microneedle array when the aqueous solution comprises the functionalized polymer, the conductive polymer and the photoinitiator; and

removing the microneedle array from the mold.

In another aspect, there is provided for a method of delivering an active agent to a subject through the device described according to the above aspect and various embodiments disclosed herein, the method comprising:

applying the microneedle array on the subject;

placing the anode and the cathode on the subject; and

operating the iontophoresis unit to deliver the active agent from the microneedle array.

### BRIEF DESCRIPTION OF THE DRAWINGS

The drawings are not necessarily to scale, emphasis instead generally being placed upon illustrating the principles of the invention. In the following description, various embodiments of the present disclosure are described with reference to the following drawings, in which:

FIG. 1A shows distribution of poly(3,4-ethylenedioxythiophene) polystyrene sulfonate (PEDOT:PSS) in a polyvinyl alcohol (PVA) matrix. The concentration of PEDOT:PSS in PVA is 5 wt %.

FIG. 1B shows uniform and homogenous dispersion of PEDOT:PSS in the MN structure. The concentration of PEDOT:PSS in a HA polymer solution used to attain this ranges from about 5 wt % to about 15 wt %.

FIG. 2A shows a design of a conductive MN array. Specifically, FIG. 2A is an optical image of the present hyaluronic acid and poly(3,4-ethylenedioxythiophene) polystyrene sulfonate (HA/PEDOT:PSS) MN array. The scale bar denotes 100 μm.

FIG. 2B shows the encapsulation of the near-infrared Cy5 dye in the conductive MN tips. The scale bar denotes 500 μm.

FIG. 2C is a confocal imaging of single MN showing the distribution of Cy5 dye within the MN tip. The scale bar denotes 100 μm.

FIG. 2D shows the micron-size holes left in the skin from penetration of the MNs (a deep blue color observed is from the PEDOT:PSS) in the epidermis layer after the insertion of conductive MNs into mice skin. The scale bar denotes 100 μm.

FIG. 3A is a plot that characterizes the present HA/PEDOT:PSS MN array in terms of load versus displacement curves for HA/PEDOT:PSS MN arrays loaded with different PEDOT:PSS concentrations.

FIG. 3B shows the hematoxylin and eosin (H&E) stain of MN-treated porcine skin. Scale bar denotes 500 μm.

FIG. 3C shows the measurement of mice skin resistance using a multimeter before and after insertion of HA MN, and conductive MN with different PEDOT:PSS concentrations.

FIG. 3D shows the current-voltage (I-V) plot comparing the curves of HA MN and HA/PEDOT:PSS MN arrays.

FIG. 3E shows the quantification of Cy5 dye penetration depth in 1.4 wt % agarose gel. (**p<0.01) for HA MN (0 mA), HA MN (3 mA), and HA with 5 wt % of PEDOT:PSS MN (3 mA).

FIG. 4 shows the combined use of iontophoresis and a conductive MN patch according to various embodiments disclosed herein.

FIG. 5 is a flow diagram showing an in vitro experimental set up for the MN-mediated iontophoretic delivery of Cy5 dye. The MNs array (1) before and (2) after insertion into 1.4 wt % agarose gel are shown. In (3), the MNs tips left inside 1.4 wt % agarose gel are shown. In (4), application of the cathode on the site of MN application is shown. In (5), iontophoresis application of a low voltage current with a current flux density of 3 mA/cm² is shown.

FIG. 6A shows distribution of Cy5 dye in 1.4 wt % agarose gel. Specifically, FIG. 6A is a two dimensional (2D) planar view of Cy5 penetration in 1.4 wt % agarose gel when treated using (i) HA MN, (ii) HA MN and iontophoresis, and (iii) HA/PEDOT:PSS MN and iontophoresis.

FIG. 6B quantifies average depth of penetration of different treatment groups (**p<0.01).

FIG. 6C is a representative 2D view of Cy5 penetration in the z-axis in samples treated using (i) HA MN and iontophoresis and (ii) HA/PEDOT:PSS MN and iontophoresis.

FIG. 7 shows two schematic diagrams. The top schematic diagram shows an in vivo model to explore the use of the present conductive MN-mediated iontophoresis for transdermal drug delivery (TDD). Mice were treated with the present drug loaded MN for 1 minute at the dorsal skin, followed by removal of the MN base patch. Next, the cathode was placed at the site of MN application and iontophoresis was performed for 3 minutes. The bottom schematic diagram breaks down in stages (i) to (iv) how the present conductive MN array delivers a drug with the use of iontophoresis.

FIG. 8A shows representative fluorescent images of skin sections stained with Hoechst (blue) and fluorescent Cy5 dye (purple) for an in vivo study. The scale bar in each of the images denotes 100μ.

FIG. 8B is the quantification of fluorescence particles count in epidermis (stratum corneum/epidermis) and dermis layer (**p<0.05).

FIG. 9A shows the experimental set-up of mice models for in vivo studies of the conductive MN and iontophoresis, according to various embodiments disclosed herein, on transdermal drug penetration.

FIG. 9B shows the representative In Vivo Imaging System (IVIS) images for experimental mice.

FIG. 9C is a quantification of fluorescence intensity from the mice models of FIG. 9B (**p<0.01).

FIG. 10A is a schematic representation of a phantom model used in the study of dye penetration through a bone sample.

FIG. 10B shows the confocal scanning microscopy images of sectioned skin tissue treated with HA MN and HA/PEDOT:PSS MN in combination with iontophoresis.

FIG. 10C shows a representative full skin section of bottom layer sample treated with conductive MN and iontophoresis. Each of the scale bars denotes 100 μm.

FIG. 11A shows the experimental set-up for in vivo drug efficacy study on a rabbit model. The experimental set-up showed how the present conductive MN and iontophoresis treatment are performed.

FIG. 11B are IVIS images showing presence of dye in the respective bone samples: (i) control and (ii) treated with the present conductive MN and iontophoresis.

FIG. 11C is a graph depicting the number of rabbits that displayed anaesthetic effect in each of the treatment procedures.

FIG. 11D is the statistical evaluation to establish efficacy of the treatment procedures in comparison to the injection group and (a) the present conductive MN and Iontophoresis, (b) topical gel, (c) the present conductive MN, and (d) iontophoresis.

FIG. 12A shows a swellable conductive MN array according to various embodiments described herein.

FIG. 12B shows the swellable conductive MN array before insertion into 1.4 wt % agarose gel.

FIG. 12C shows the swellable conductive MN array after insertion into 1.4 wt % agarose gel.

FIG. 12D shows micron-size holes created on agarose gel.

FIG. 12E shows the tips of swellable conductive MN array exhibiting swollen morphology.

FIG. 12F shows the swellable conductive MN array returning to its original conformation 24 hours after removal.

FIG. 13 shows the swelling behaviors of crosslinked MeHA-MN patches studied in 1.4 wt % agarose gel after 1 minute of incubation.

FIG. 14 shows the representative images of CL5-MeHA MN patches before and after the loading of FITC, FITC-Dextran and Doxorubicin. Scale bar denotes 2 mm.

FIG. 15 shows the release profiles of FITC, FITC-Dextran, and Doxorubicin from MeHA MN patches in the first 1 hour.

FIG. 16 shows a comparison of conventional needle and syringe approach against the present conductive MN array used with iontophoresis, wherein the conventional approach has several limitations that contribute to increased patient anxiety resulting in a time-consuming administration.

FIG. 17 illustrates fabrication of the present MN patches with a flexible base and stiff microneedles, including fabrication of the template for forming the MNs.

FIG. 18A shows the fabrication of a supporting substrate for producing the MN patch.

FIG. 18B shows the fabrication of MNs for the MN patch.

FIG. 19A shows a typical SEM image of a microneedle device formed from poly(ethylene glycol) diacrylate (PEGDA) according to various embodiments disclosed herein.

FIG. 19B shows the elastic modulus and hardness of the microneedle device of FIG. 19A.

FIG. 19C is a mechanical compression test for the microneedle device of FIG. 19A.

FIG. 20A is an illustration of a fresh porcine skin surface after penetration with the MN device (microneedle patch). Scale bar denotes 5 mm.

FIG. 20B shows a histological picture of the porcine skin after being applied with the microneedle patch. Scale bar denotes 200 μm.

FIG. 21 is a characterization of the dye-loaded microneedle patches. Bright-field and fluorescent microscopy images of a microneedle patch fabricated with a HA-based supportive matrix and having fluorescence dye Cy5-loaded solid HA-tips. Each of the scale bars denotes 100 μm. The top row of images show the side-view of the MN patch. The bottom row of images shows the top-down view of the MN patch.

FIG. 22A is a characterization of the drug-loaded PLGA based MNs-array patch, wherein close-up images of the MNs-array patch.

FIG. 22B show the bright-field and confocal microscopy images of the MNs-array patch fabricated with a HA-based supportive matrix and fluorescence dye solid PLGA-tips of FIG. 22A.

FIG. 23A shows a photograph of a flexible free-standing polypyrrole (PPy) nanotube film.

FIG. 23B shows a photograph of the flexible free-standing polypyrrole (PPy) nanotube film of FIG. 23A in its bent state.

FIG. 23C is a plot of electrical conductivity against temperature for the flexible free-standing PPy nanotube film of FIG. 23A.

FIG. 24 shows a schematic representation of drug delivery using swellable conductive MNs and iontophoresis.

FIG. 25A is a schematic of the fabrication process of crosslinked MeHA MNs.

FIG. 25B is a plot of the swelling ratio at different times.

FIG. 25C shows optical images of crosslinked MeHA MNs before and after maximum swelling. Scale bars denote 1 mm.

### DETAILED DESCRIPTION

The following detailed description refers to the accompanying drawings that show, by way of illustration, specific details and embodiments in which the invention may be practised. These embodiments are described in sufficient detail to enable those skilled in the art to practice the invention. Other embodiments may be utilized and changes may be made without departing from the scope of the invention. The various embodiments are not necessarily mutually exclusive, as some embodiments can be combined with one or more other embodiments to form new embodiments.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

Various embodiments of the present disclosure relate to a microneedle (MN) patch for delivery of an active agent to, for example, the sensory nerves for various applications, such as but not limited to, oral and maxillofacial surgery. The active agent may be an anaesthetic agent or a therapeutic drug. The microneedle patch may comprise an array of microneedles and hence termed herein a microneedle array. The microneedle array is conductive, and this means it is electrically conductive in the context of the present disclosure. The conductive MN array may have microneedles developed with a double-layered structure composed of a biocompatible polymer and a conductive polymer, or a matrix comprising the biocompatible polymer with the conductive polymer incorporated therein. When the MN array is combined with iontophoresis for use, the combination advantageously allows control and enhances permeation of drugs, such as local anaesthetic (lidocaine), to and/or through the layers in skin, mucosa, and/or cortical bone to reach, for instance, the nerves resulting in a numbing effect.

Iontophoresis is an efficient and painless method of rapidly delivering therapeutics to a localized tissue area by using electrical current. A device comprising an iontophoresis unit may comprise two electrodes and a power supply. The drug formulation may be placed on one of the electrodes while the other may contain only a reference gel.

The combination of MN and iontophoresis renders a minimally-invasive and fast delivery of anaesthetics, in dental practice as one of the non-limiting examples.

Particularly, a conductive microneedle (MN) array for iontophoretic delivery of anaesthetic agents into the oral mucosa and underlying alveolar bone to target the sensory nerves supplying teeth has been described herein as one example of applying the conductive MN array. The conductive microneedles may be fabricated to have a length in the range of, e.g. 150 μm to 200 μm, for painless penetration of the oral epithelium without contacting nerve endings in the lamina propria while creating micro-conduits for delivery of drugs into the oral tissue. Additionally, iontophoresis provides a low-voltage current as a driving force for accelerating drug penetration to the nerves in the alveolar bone that supply sensation to the teeth. The conductive property of the MNs significantly lowers resistance of the oral mucosa, giving rise to more drug molecules delivered quicker into deeper tissues. The conductive MN patch, used in combination with iontophoresis, showed almost immediate dental anaesthetic effect in a rabbit model. This is expected to eliminate patients' phobia of dental anaesthesia delivery, promote patient compliance in seeking timely dental treatments and reduce a nation's oral disease burden. Dentists may also save time spent on behavioural management of phobic patients, improving clinic efficiency that translates to overall cost-savings.

To further demonstrate one advantage of the MN array disclosed herein, the application of a MN array having microneedles 100 μm to 150 μm long in delivery of anaesthetics, instead of hypodermic needles or syringes, is discussed. In such instance, the patient does not feel any pain from application of the 100 μm to 150 μm long MNs on the gum, which significantly eliminates anxiety and fear. The reduction of drug (e.g. anaesthetics) release and/or delivery time is also enhanced. The typical waiting time for injection and diffusion of anaesthetics to achieve desired numbness is typically 5 minutes or more, which increases patient anxiety. In comparison, the present conductive MN array when used with iontophoresis significantly reduce time taken for the drug delivery process to less than 1 minute. As already mentioned above and discussed herein, the conductive polymer in the skin reduces the skin's resistance and increases the electromotive force passing through the skin. This leads to enhanced efficacy in drug delivery and potentially result in a reduction of anaesthetic dosage.

The MN array of the present disclosure may have a base with the microneedles disposed thereon. The microneedles may be formed of (i) a swellable and water-insoluble matrix or (ii) a water-soluble matrix.

With the above in mind, details of the MN array, a device and method which include the MN array for delivering the active agent, their uses thereof, and a method of producing the MN array, and their various embodiments, are described as follow.

In the present disclosure, there is provided for a microneedle array comprising a base having microneedles disposed thereon, wherein each of the microneedles may be formed of (i) a swellable and water-insoluble matrix comprising a crosslinked polymer or (ii) a water-soluble matrix comprising a water-soluble polymer, and a conductive polymer incorporated in the swellable and water-insoluble matrix or the water-soluble matrix. The crosslinked polymer may comprise or may be a hydrophobic polymer functionalized with one or more functional groups, non-limiting examples of which may include carboxyl groups, hydroxyl groups, etc., that aid in formation of the crosslinked polymer. The crosslinked polymer may comprise or may also be a crosslinked hydrophilic polymer. The crosslinked hydrophilic polymer may have one or more of the functional groups mentioned above. The water-soluble polymer may have one or more carboxyl or hydroxyl groups.

The term “swellable” used herein means that a material can increase in size by absorbing substances such as, but not limited to, biological fluids. A non-limiting example of a biological fluid is water. The swellable material, after having its size increased, may return to its original size and/or shape. The matrix which the microneedles are formed of may be a swellable matrix.

The term “water-insoluble” used herein refers to a material that does not dissolve in an aqueous medium. An example of the aqueous medium may be water. The swellable matrix which the microneedles are formed of may be a water-insoluble matrix, and accordingly termed a “swellable and water-insoluble matrix”.

In embodiments where the microneedles are formed of the swellable and water-insoluble matrix, the swellable and water-insoluble matrix may comprise or may be formed of a crosslinked polymer. A crosslinked polymer herein refers to a polymer having an internal network of bonds that link one or more chains of the polymer. The bonds may include covalent bond, ionic bond, hydrogen bond, etc. The crosslinked polymer may comprise one or more hydroxyl (—OH) groups. The crosslinked polymer may be a hydrophilic polymer and hence referred to as a crosslinked hydrophilic polymer. The crosslinked hydrophilic polymer may comprise one or more hydroxyl groups. The one or more hydroxyl groups advantageously allow for linkages to be formed between the polymer chains via crosslinkers. Such linkages may constitute an internal network of the crosslinked polymer, such that an active agent may be encapsulated in the network forming the matrix and released therefrom when the matrix swells. The active agent may be a therapeutic drug, an anaesthetic agent, or any other active agent that is to be delivered in such manner.

The crosslinked polymer may comprise or may be formed of an acrylate-crosslinked hydrophilic polymer, a furan-crosslinked hydrophilic polymer, or a catechol-crosslinked hydrophilic polymer. In other words, the crosslinker for crosslinking the hydrophilic polymer may be an acrylate-based compound, a furan-based compound, or a compound having at least one catechol group. The acrylate-based compound may be a methacrylate-based compound, and accordingly, the acrylate-crosslinked hydrophilic polymer may be or may comprise a methacrylate-crosslinked hydrophilic polymer. A non-limiting example of the acrylate-based compound may be methacrylic anhydride. A non-limiting example of the furan-based compound may be furan. A non-limiting example of the catechol-based compound may be catechol. Other crosslinkers that can form a network of bonds that links the polymer chains to impart swellability to the matrix may be used. Such crosslinkers may be applied on a hydrophobic polymer to form the crosslinked polymer.

In the present disclosure, the acrylate-crosslinked hydrophilic polymer may comprise or may consist of methacrylate-crosslinked hyaluronic acid, methacrylate-crosslinked polyvinyl alcohol, methacrylate-crosslinked poly(methylvinyl ether), or crosslinked poly(ethylene glycol) diacrylate. The methacrylate-crosslinked hyaluronic acid may be formed from hyaluronic acid having an average molecular weight ranging from 3 kDA to 300 kDa, 50 kDa to 300 kDa, 100 kDa to 300 kDa, 150 kDa to 300 kDa, 200 kDa to 300 kDa, 250 kDa to 300 kDa, etc. Other polymers used to form the crosslinked polymers may have an average molecular weight of the specified ranges. Such average molecular weights provide sufficient viscosity for a polymer to be filled into a mold and subsequently crosslinked to form the microneedle array. If the polymer used to form the microneedle array, such as the microneedles, is too viscous or not sufficiently viscous, the polymer may not properly fill into the mold for forming the microneedle array.

In embodiments where the microneedles are formed of a water-soluble matrix, the water-soluble matrix may comprise or may be formed of a water-soluble polymer. The term “water-soluble” used herein refers to a material that can dissolve in an aqueous medium. An example of the aqueous medium may be water. The water-soluble matrix which the microneedles are formed of may be a water-soluble matrix.

The water-soluble polymer may have one or more hydroxyl groups. Advantageously, the one or more hydroxyl groups may help in and/or increase dissolution, faster and/or more, of the water-soluble polymer in an aqueous medium, e.g. water. Dissolution of a matrix comprising or formed of such water-soluble polymer having one or more hydroxyl groups allows for an active agent to be encapsulated therein and released therefrom when the matrix dissolves. The active agent may be a therapeutic drug, an anaesthetic agent, or any other active agent that is to be delivered in such manner.

The water-soluble polymer may comprise hyaluronic acid, polyvinyl alcohol, poly(methylvinyl ether), poly(ethylene glycol), or poly(lactic-co-glycolic acid). The hyaluronic acid may have an average molecular weight ranging from 3 kDa to 300 kDa, 50 kDa to 300 kDa, 100 kDa to 300 kDa, 150 kDa to 300 kDa, 200 kDa to 300 kDa, 250 kDa to 300 kDa, etc. Such average molecular weights provide sufficient viscosity for the water-soluble polymer to be filled into a mold and subsequently dried to form the microneedle array. If the polymer used to form the microneedle array, such as the microneedles, is too viscous or not sufficiently viscous, the polymer may not properly fill into the mold for forming the microneedle array.

Regardless of the microneedle array having microneedles formed of the swellable and water-insoluble matrix or the water-soluble matrix, various embodiments of the microneedle array include a conductive polymer incorporated in the swellable and water-insoluble matrix or the water-soluble matrix. The term “conductive” used herein with respect to a conductive polymer refers to an electrically conductive polymer. For instance, if the present disclosure indicates a polymer is conductive, it means that the polymer conducts electricity. The conductive polymer not only renders the microneedle array compatible with iontophoresis to enhance delivery of the active agent, but also reduces resistance of the surface layers which the microneedle array is applied on (e.g. skin tissue including stratum corneum or mucous layer) to enable stronger flow of charges. With stronger flow of charges, the active agent, whether charged or uncharged, experiences a stronger driving force and gets delivered faster and/or more.

The conductive polymer may comprise up 25 wt % or less, such as 1 wt % to 20 wt %, 5 wt % to 20 wt %, 10 wt % to 20 wt %, 15 wt % to 20 wt %, 5 wt % to 15 wt %, 5 wt % to 10 wt %, etc. of the swellable and water-insoluble matrix or the water-soluble matrix. Such amounts of conductive polymer render a homogenous distribution throughout the matrix when the conductive polymer is doped therein. Otherwise, the conductive polymer may agglomerate in the matrix and disrupt fabrication of the microneedle array. For instance, if particles of the conductive polymer are present, solvent casting may not be used effectively to form the microneedle array. The term “doped” and grammatical variants thereof are used interchangeably with the term “incorporated” and its grammtical variants.

The conductive polymer may comprise or consist of poly(3,4-ethylenedioxythiophene)-poly(styrenesulfonate), polypyrrole, polyaniline, polythiophene, polyethyne, poly(p-phenylene), or poly(p-phenylene vinylene). Other conductive polymers that can impart the advantages mentioned above and compatible with the material forming the matrix may be used.

The microneedle array has microneedles formed on a base. The base may be a rigid base or a flexible base. A rigid base, if subject to any form of contortion, may become damage or unable to revert to its original conformation on its own. The term “flexible” used herein means that the material may be subjected to any form of contortion during use, including bending, twisting, tension and compression, without getting damage and can revert to its original conformation independently.

The base may comprise or may consist of the crosslinked polymer or the water-soluble polymer which the swellable and water-insoluble matrix or the water-soluble matrix is respectively formed of. The base may comprise or consist of a different crosslinked polymer or the water-soluble polymer from that used to form the matrix.

The microneedles may extend away from the base. The microneedles may have a length of 1000 μm or less. In other words, each of the microneedles may have a length of 1000 μm or less, 900 μm or less, 800 μm or less, 700 μm or less, 600 μm or less, 500 μm or less, 400 μM or less, 300 μm or less, 200 μm or less, 100 μm or less. All the microneedles may have the same length. The microneedles may all have a length ranging from 100 μM to 700 μM or 100 μm to 150 μM, etc. The present microneedles are versatile in that the length of the microneedles may be designed based on where the active agent is intended to be delivered to in a subject. For deeper penetration of the active agent, the microneedle may have the longer range of length specified above. For penetrating into surfaces and tissue layers that are thinner so as to avoid the MNs contacting the nerves which may likely cause pain, the microneedle may have the shorter range of length specified above.

Various embodiments of the microneedle array may further include an active agent. The base my have a surface for the active agent to be disposed thereon and/or the swellable and water-insoluble matrix or the water-soluble matrix further comprises the active agent disposed therein. For example, the active agent may be disposed on the surface of the microneedles, and this may include being disposed only at the tip of the microneedles. The active agent may be loaded into tips of the microneedles. The active agent may be disposed on a surface of the base which the microneedles do not extend from, for example, the active agent may be in the form of a hydrogel or encapsulated in a hydrogel that is attachable to or placeable onto the base. In such configuration, the active agent may be driven from the base through the microneedles into a subject which the microneedle array is applied to by iontophoresis. Said differently, the active agent may be separately applied before and/or after the microneedle array is applied on a subject. In other non-limiting example, the base made of the crosslinked hydrophilic polymer may be used to contain the active agent. The active agent may be applied directly on the subject after applying the microneedle array.

The present disclosure also provides for a device operable or configured to deliver an active agent. The device may comprise a microneedle array, wherein the microneedle array may comprise a base having microneedles disposed thereon, wherein each of the microneedles is formed of (i) a swellable and water-insoluble matrix comprising a crosslinked polymer or (ii) a water-soluble matrix comprising a water-soluble polymer, and a conductive polymer incorporated in the swellable and water-insoluble matrix or the water-soluble matrix, and an iontophoresis unit comprising an anode and a cathode connectable to the microneedle array, wherein the iontophoresis unit is operable to deliver the active agent from the microneedle array. Embodiments and advantages described in the context of the present microneedle array are analogously valid for the present device as described herein, and vice versa. Embodiments and advantages of the microneedle array have already been mentioned above and demonstrated in the examples, and shall not be iterated for brevity. For instance, as already mentioned above, the crosslinked polymer may be a hydrophilic polymer or a hydrophobic polymer. The hydrophilic polymer and hydrophobic polymer may be capable of being crosslinked to form the crosslinked polymer.

In embodiments where the microneedles are formed of the swellable and water-insoluble matrix, the crosslinked polymer may comprise an acrylate-crosslinked hydrophilic polymer, a furan-crosslinked hydrophilic polymer, or a catechol-crosslinked hydrophilic polymer. The acrylate-crosslinked hydrophilic polymer may comprise methacrylate-crosslinked hyaluronic acid, methacrylate-crosslinked polyvinyl alcohol, methacrylate-crosslinked poly(methylvinyl ether), or crosslinked poly(ethylene glycol) diacrylate. The methacrylate-crosslinked hyaluronic acid may be formed from hyaluronic acid having an average molecular weight ranging from 3 kDa to 300 kDa, 50 kDa to 300 kDa, 100 kDa to 300 kDa, 150 kDa to 300 kDa, 200 kDa to 300 kDa, 250 kDa to 300 kDa, etc.

In embodiments where the microneedles are formed of the water-soluble matrix, the water-soluble polymer may comprise hyaluronic acid, polyvinyl alcohol, poly(methylvinyl ether), poly(ethylene glycol), or poly(lactic-co-glycolic acid). The hyaluronic acid may have an average molecular weight ranging from 3 kDa to 300 kDa, 50 kDa to 300 kDa, 100 kDa to 300 kDa, 150 kDa to 300 kDa, 200 kDa to 300 kDa, 250 kDa to 300 kDa, etc.

In various embodiments, the conductive polymer may comprise 25 wt % or less, e.g. 1 wt % to 20 wt %, 5 wt % to 20 wt %, 10 wt % to 20 wt %, 15 wt % to 20 wt %, 5 wt % to 15 wt %, 5 wt % to 10 wt %, etc. of the swellable and water-insoluble matrix or the water-soluble matrix. The conductive polymer may comprise poly(3,4-ethylenedioxythiophene)-poly(styrenesulfonate), polypyrrole, polyaniline, polythiophene, polyethyne, poly(p-phenylene), or poly(p-phenylene vinylene).

As already mentioned above, each of the microneedles may have a length of 1000 μm or less, 900 μm or less, 800 μm or less, 700 μm or less, 600 μm or less, 500 μm or less, 400 μm or less, 300 μm or less, 200 μm or less, 100 μm or less. All the microneedles may have the same length. The microneedles may all have a length ranging from 100 μm to 700 μm or 100 μm to 150 μM, etc. Such lengths render the microneedles mechanically advantageous for application onto a subject, e.g. penetrating the dermal layer, deep dermis layer, and/or mucosa, of the subject. If the microneedles are to be longer, there may be a risk that the microneedles become insufficiently rigid for penetration.

In various embodiments, the base may comprise the crosslinked polymer or the water-soluble polymer which the swellable and water-insoluble matrix or the water-soluble matrix is respectively formed of. The base may comprise or may be formed of a crosslinked polymer or a water-soluble polymer different from that used to form the matrix. The base may have a surface for the active agent to be disposed thereon, and/or the swellable and water-insoluble matrix or the water-soluble matrix may further comprise the active agent disposed therein. Examples of how the active agent may be disposed on the microneedle array have already been discused above.

The active agent may comprise an anaesthetic agent and/or any drug. The drug may be a therapeutic drug.

The present disclosure also provides for a method of producing a microneedle array, wherein the microneedle array may comprise a base having microneedles disposed thereon, wherein each of the microneedles is formed of (i) a swellable and water-insoluble matrix comprising a crosslinked polymer or (ii) a water-soluble matrix comprising a water-soluble polymer, and a conductive polymer incorporated in the swellable and water-insoluble matrix or the water-soluble matrix. The method may comprise providing an aqueous solution in a mold, wherein the aqueous solution comprises (i) a functionalized polymer, the conductive polymer and a photoinitiator, or (ii) the water-soluble polymer and the conductive polymer, irradiating the aqueous solution to form the microneedle array when the aqueous solution comprises the functionalized polymer, the conductive polymer and the photoinitiator, and removing the microneedle array from the mold. Embodiments and advantages described in the context of the present microneedle array and the present device are analogously valid for the present method as described herein, and vice versa. Embodiments and advantages of the microneedle array and device have already been mentioned above and demonstrated in the examples, and shall not be iterated for brevity. For instance, as already mentioned above, the crosslinked polymer may be a hydrophilic polymer or a hydrophobic polymer. The hydrophilic polymer and hydrophobic polymer may be capable of being crosslinked to form the crosslinked polymer.

In the present method, providing the aqueous solution may comprise dissolving the functionalized polymer or the water-soluble polymer in an aqueous medium, such as water. In embodiments where microneedles are to be formed of the swellable and water-insoluble matrix, the functionalized polymer is used. In embodiments where microneedles are to be formed of the water-soluble matrix, the water-soluble polymer is used.

In various embodiments, providing the aqueous solution may comprise dissolving the functionalized polymer or the water-soluble polymer in water at a concentration ranging from 25 mg/mL to 100 mg/mL, 50 mg/mL to 100 mg/mL, 75 mg/mL to 100 mg/mL, 25 mg/mL to 50 mg/mL, 25 mg/mL to 75 mg/mL, or 50 mg/mL to 75 mg/mL, etc. Advantageously, such concentrations provide sufficient viscosity for the aqueous solution to be filled into the mold for forming the microneedle array. If the concentration is lower or higher, there may be a risk that the aqueous solution may not dry completely for the microneedles to form properly and/or not enough polymer to even form the matrix, or the aqueous solution may become too viscous and not fill the mold properly, respectively. The concentration range may depend on the functionalized polymer used. The concentration range may depend on the water-soluble polymer used.

The functionalized polymer may comprise or may be a hydrophilic polymer or a hydrophobic polymer. Such functionalized hydrophilic or hydrophobic polymer may have one or more functional groups, non-limiting examples of which may include carboxyl groups, hydroxyl groups, etc., that aid in formation of the crosslinked polymer. The functionalized polymer may comprise or may consist of an acrylate-functionalized hydrophilic polymer, a furan-functionalized hydrophilic polymer, or a catechol-functionalized hydrophilic polymer. Non-limiting examples of these polymers have already been discussed above. For instance, the acrylate-functionalized hydrophilic polymer may comprise methacrylate-functionalized hyaluronic acid, methacrylate-functionalized polyvinyl alcohol, methacrylate-functionalized poly(methylvinyl ether), or diacrylate-functionalized poly(ethylene glycol).

The functionalized polymer may be prepared by functionalizing the hydrophilic or hydrophobic polymer with a functional group for forming the crosslinked polymer. The functional group may be imparted onto the hydrophilic or hydrophobic polymer when the hydrophilic or hydrophobic polymer is reacted with a compound containing the functional group. For example, to obtain methacrylate-functionalized hyaluronic acid, the hydrophilic polymer of hyaluronic acid may be mixed with methacrylate anhydride. To obtain furan-functionalized or catechol-functionalized hydrophilic polymer, the hydrophilic polymer may be reacted with a furan-based compound or a compound having at least one catechol group, respectively. The functionalized hydrophilic polymer may be subsequently crosslinked via the functional group(s) present thereon.

In embodiments where the water-soluble polymer are used, the water-soluble polymer may comprise hyaluronic acid, polyvinyl alcohol, poly(methylvinyl ether), poly(ethylene glycol), or poly(lactic-co-glycolic acid). The water-soluble polymer may have one or more carboxyl or hydroxyl groups.

In the present method, providing the aqueous solution may comprise (i) mixing the functionalized polymer with the conductive polymer and a photoinitiator or (ii) mixing the water-soluble polymer with the conductive polymer. The photoinitiator is used to aid crosslinking of the functionalized hydrophilic polymer via the functional groups present thereon in the presence of light. This means that crosslinking of the functional groups, such as the methacrylate, furan, or catechol functional groups, get activated in the presence of the photoinitiator and light to convert the functionalized polymer to the crosslinked polymer. Non-limiting examples of the photoinitiator may include diethoxyacetophenone (DEAP), dimethoxyphenylacetophenone, benzoylcyclohexanol, or hydroxydimethylacetophenone.

In the present method, the conductive polymer may be mixed at a concentration of 25 wt % or less, such as ranging from 1 wt % to 20 wt %, 5 wt % to 20 wt %, 10 wt % to 20 wt %, 15 wt % to 20 wt %, 5 wt % to 15 wt %, 5 wt % to 10 wt %, etc. of the aqueous solution.

The conductive polymer may comprise or may consist of poly(3,4-ethylenedioxythiophene)-poly(styrenesulfonate), polypyrrole, polyaniline, polythiophene, polyethyne, poly(p-phenylene), or poly(p-phenylene vinylene).

In the present method, the mold may comprise a plurality of cavities shaped to form the microneedles. The plurality of cavities are not limited to pyramidal shapes, but can be tubular, frustoconical, conical, or other shapes penetrable into, for example, a dermal layer, a mucosa layer, an oral epithelium, deep dermis layer, bone, etc. The design of the cavities determines the design (e.g. shape) of the microneedles.

Each of the plurality of cavities has a depth of 1000 μm or less, 900 μm or less, 800 μm or less, 700 μm or less, 600 μm or less, 500 μm or less, 400 μm or less, 300 μm or less, 200 μm or less, 100 μm or less. All the cavities may have the same depth. The cavities may all have a depth ranging from 100 μM to 700 μm or 100 μm to 150 μm, etc.

The present method may further comprise centrifuging the mold with the aqueous solution provided therein. Where the swellable and water-insoluble matrix is to be formed, the centrifuging may be carried out prior to irradiating the aqueous solution. The centrifuging advantageously ensures the cavities are completely filled with the aqueous solution so as to have the microneedles properly formed.

The present disclosure provides for a method of delivering an active agent to a subject through the device as described above and herein. The method may comprise applying the microneedle array on the subject, placing the anode and the cathode on the subject, and operating the iontophoresis unit to deliver the active agent from the microneedle array. Embodiments and advantages described in the context of the present microneedle array, the present device and the present method of producing the microneedle array, are analogously valid for the present method of delivering the active agent as described herein, and vice versa. Embodiments and advantages of the microneedle array, the device, and the present method of producing the microneedle array, have already been mentioned above and demonstrated in the examples, and shall not be iterated for brevity.

In various embodiments, applying the microneedle array may comprise inserting the microneedles into a first surface of the subject. The first surface may be the skin of the subject. The first surface may be a surface of a dermal layer, a mucosa layer, a bone, etc.

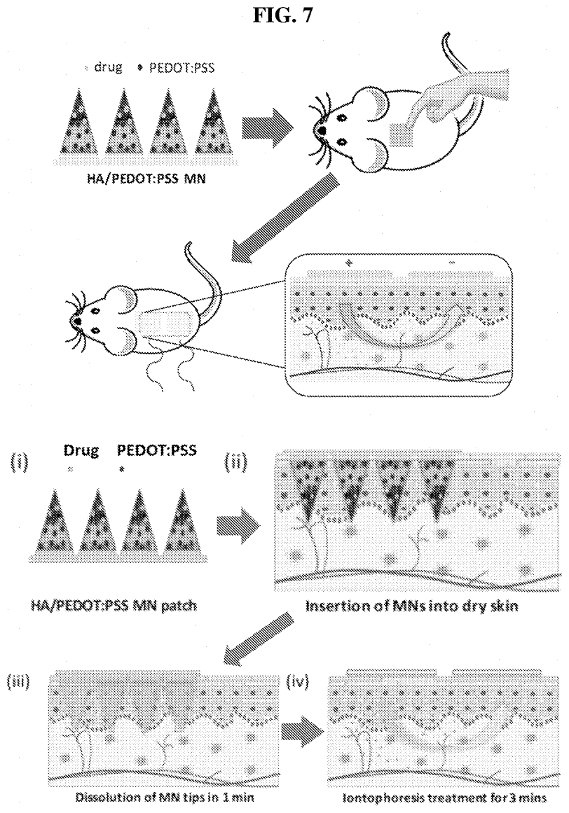

In the present method, placing the anode and the cathode on the subject may comprises (i) arranging the anode on the first surface proximal to where the microneedle may be applied and arranging the cathode on a second surface distal to where the anode may be arranged when the active agent is anionic, or (ii) arranging the cathode on the first surface proximal to where the microneedle may be applied and arranging the cathode on a second surface distal to where the cathode may be arranged when the active agent is cationic, or (iii) arranging either the anode or the cathode on the first surface proximal to where the microneedle may be applied and arranging the cathode or the anode, respectively, on a second surface to where the anode or cathode may be arranged, respectively, when the active agent is neutral. For example, the microneedles can be first applied on the buccal surface (an example of first surface) of the gum even though the drug is targeted for delivery first through the mucosa, then into and through the bone to the nerves. Depending whether the drug is positively charged, negatively charged, or neutral, the anode or cathode can be placed onto the microneedle base or directly over the area where the microneedle array was applied while the other electrode may be placed on the opposing surface, in this instance, the lingual surface (example of the second surface) of the gum, along the same plane. Advantageously, the anode and cathode may be positioned on the subject in any manner as long as iontophoresis can be carried out to drive faster and/or more delivery of the drug to the target area. This demonstrates how versatile the present method is. Other non-limiting examples of how the anode and cathode may be placed are shown in the figures, for instance, FIG. 4 and FIG. 7.

In the present method, operating the iontophoresis unit may comprise passing an electrical current between the anode and the cathode to establish a voltage for delivering the active agent from the microneedle array. The current and voltage applied may be controlled at a level that does not cause discomfort, or even pain, to the subject.

In the present method, delivering the active agent from the microneedle array may comprise delivering the active agent to and/or through (i) a dermal layer of the subject, and/or (ii) a mucosa of the subject, and/or (iii) a deep dermis layer of the subject, and/or (iv) a bone of the subject. These are non-limiting examples of where the drug may be delivered via the present microneedle array, present device and present method.

In the context of the present disclosure, the word “substantially” does not exclude “completely” e.g. a composition which is “substantially free” from Y may be completely free from Y. Where necessary, the word “substantially” may be omitted from the definition of the invention.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance. The variance may be ±0.1%, ±0.5%, ±1%, ±5%, or even ±10%.

As used herein, the term “and/or” includes any and all combinations of one or more of the associated listed items.

Unless specified otherwise, the terms “comprising” and “comprise”, and grammatical variants thereof, are intended to represent “open” or “inclusive” language such that they include recited elements but also permit inclusion of additional, unrecited elements.

While the methods described above are illustrated and described as a series of steps or events, it will be appreciated that any ordering of such steps or events are not to be interpreted in a limiting sense. For example, some steps may occur in different orders and/or concurrently with other steps or events apart from those illustrated and/or described herein. In addition, not all illustrated steps may be required to implement one or more aspects or embodiments described herein. Also, one or more of the steps depicted herein may be carried out in one or more separate acts and/or phases.

### EXAMPLES

The present disclosure provides for a microneedle (MN) array that is at least electrically conductive.

The MN array disclosed herein, and in the examples below, is a conductive MN array using polymers, for example, hyaluronic acid (HA) and poly(3,4-ethylenedioxythiophene) polystyrene sulfonate (PEDOT:PSS). These materials have been approved by the U.S. Food and Drug Administration for a wide range of biomedical applications. The conductive MN array used together with iontophoresis provide synergistic effects in iontophoretic drug delivery to achieve deep penetration of drug molecules in a few minutes by significantly modulating skin resistance. Particularly, two different types of HA polymers have been used to demonstrate the conductive MN array.

HA is a natural and biocompatible non-sulfated glycosaminoglycan that has natural supreme hydrating functions with an ability to bind a large volume of water content. In its innate nature, HA can be used to fabricate water-dissolvable MNs.

Alternatively, covalent crosslinking of HA can be performed by modifying the hydroxyl or carboxyl groups of HA with functional moieties to result in a stable internal network enabling HA molecules to absorb fluid without dissolving. As disclosed herein, HA is crosslinked with methacrylate to produce MeHA (methacrylate-crosslinked HA) for fabricating swellable MNs.

The combined use of MeHA and PEDOT:PSS polymers, as a non-limiting example, in the fabrication of a conductive MN array are discussed below. The combined use of HA and PEDOT:PSS polymers, as another non-limiting example, in the fabrication of a conductive MN array are discussed below.

In the non-limiting examples below, it is shown that the doping of PEDOT:PSS polymer within the matrix of HA and MeHA polymers forms a conductive polymeric MN array. The use of HA and PEDOT:PSS in the fabrication of a conductive MN results in a dissolvable conductive MN array that immediately disintegrates upon insertion into the oral mucosa.

Meanwhile, the use of MeHA and PEDOT:PSS forms a conductive swellable MN array that swells within the oral mucosa.

Advantageously, the dissolvable conductive MN has shown to be effective in the delivery of anaesthetic molecules to the rabbit incisors. The efficacy of drug delivery using a dissolvable conductive MN with iontophoresis was determined to be equivalent to the traditional method of needle and syringe delivery at a 95% confidence interval.

Further advantageously, the swellable conductive MN and its combined use with iontophoresis results in a stronger electro-osmotic flow, rendering quicker migration of drug molecules from the site of MN insertion into the nerves residing within the alveolar bone.

While PEDOT:PSS doping increases electrical conductivity, the difficulty lies in the fabrication of a MN array when HA and MeHA are doped with PEDOT:PSS. FIGS. 1A and 1B show a comparison of PEDOT:PSS poorly and uniformly dispersed in PVA matrix and HA matrix, that are respectively obtained. The distribution of PEDOT:PSS in the PVA matrix of FIG. 1A shows poor dispersion when the two polymers are mixed in a random ratio, demonstrating that doping of PEDOT:PSS in a polymer is not straightforward.

Further details of the present MN array, a device comprising the MN array, their uses, and method of fabricating the MN array, are discussed, by way of non-limiting examples, as set forth below.

### Example 1A: Fabrication of Dissolvable Conductive HA/PEDOT:PSS MN Array

The HA/PEDOT:PSS MN array was fabricated using soft lithography techniques, such as micromolding, to incorporate a conductive polymer, e.g. PEDOT:PSS, in another polymer, low molecular weight HA.

Firstly, an inverse-replicate polydimethylsiloxane (PDMS) micromold was made from a pyramidal MN stainless steel master structure consisting of 100 needles in a 10×10 array with a height of about 700 μm, tip radius of about 5 μm and a base width of about 300 μm (Micropoint Technologies Pte Ltd, Singapore). This was achieved by pouring PDMS (10:1 w/w ratio of PDMS polymer to curing agent) over the MN master structure before degassing in a vacuum oven. Curing was then carried out at 70° C. for 2 hours and then peeled off from the stainless steel mold. The obtained PDMS-micromold was then repeatedly used in the fabrication of the HA/PEDOT:PSS MNs.

To fabricate the MN array, low molecular weight HA powder was dissolved in distilled water to obtain a viscous solution of HA polymer solution (0.5 g/ml) and not form HA hydrogel. The solution was then centrifuged at 10,000 rpm for 10 minutes to remove air bubbles. To increase conductivity of the HA polymer, PEDOT:PSS particles were dispersed in the HA polymer solution. The concentration of PEDOT:PSS added into the HA polymer solution ranged from 0 wt % to 25 wt %. To ensure uniform and homogenous distribution of PEDOT:PSS particles in the HA polymer matrix, the solution was sonicated continuously for 30 minutes in slow speed. Following sonication, the final mixture was centrifuged at 10,000 rpm for 10 minutes to ensure no PEDOT:PSS particulates nor residual air bubbles are present, which is for preparing a uniform polymer mixture containing both HA and PEDOT:PSS for solvent casting to prepare MN structures.

The mixture containing HA doped with PEDOT:PSS polymer is then added onto a plasma-treated PDMS MN mold and centrifuged in a swinging bucket rotor (SCANSPEED 1580R, LaboGene) at a speed of 4,000 rpm for 5 minutes to ensure that the MN tips are filled. The excess solution remaining in the mold surface was removed using a glass slide. After overnight drying at room temperature (e.g. 25° C.), a second layer of HA solution (e.g. 3 kDa to 10 kDa) was added to create the back layer of the MN patch. The back layer MN patch was air dried overnight and the entire MN array, including the back layer, was gently peeled off from the PDMS mold, which was then stored at 4° C. in an un-humidified condition.

In other examples, microneedles with a uniform height in the range of 100 μm to 700 μm were fabricated. Shorter microneedles having a uniform height in the range of 100 μm to 200 μm, 100 μm to 150 μm, 150 μm to 200 μm, etc. can also be fabricated. The microneedles' tips are not limited to pyramidal shapes, but can be tubular, frustoconical, conical, or other shapes penetrable into, for example, a dermal layer, a mucosa layer, and/or an oral epithelium. The base width can be in the range of, for example, 100 μm to 500 μm. The shorter MNs (100 μm to 200 μm) can be used to pierce the oral epithelium whilst reducing stimulation of nerves in the lamina propria. With the low molecular weight HA forming the matrix of the MN tips, MN tips that dissolve rapidly upon contact with the skin and/or mucosa interstitial fluid can provide for bolus release of drug molecules encapsulated therein.

### Example 1B: Characterization of Dissolvable Conductive HA/PEDOT:PSS MN Array

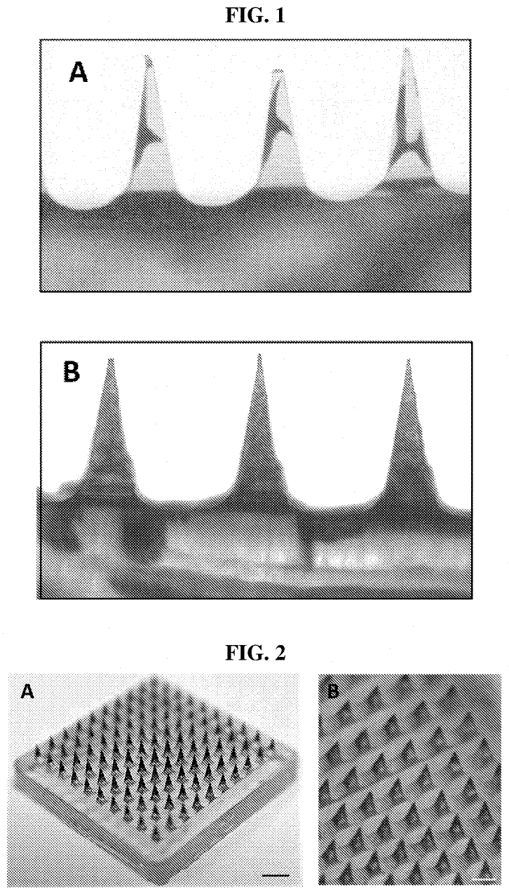

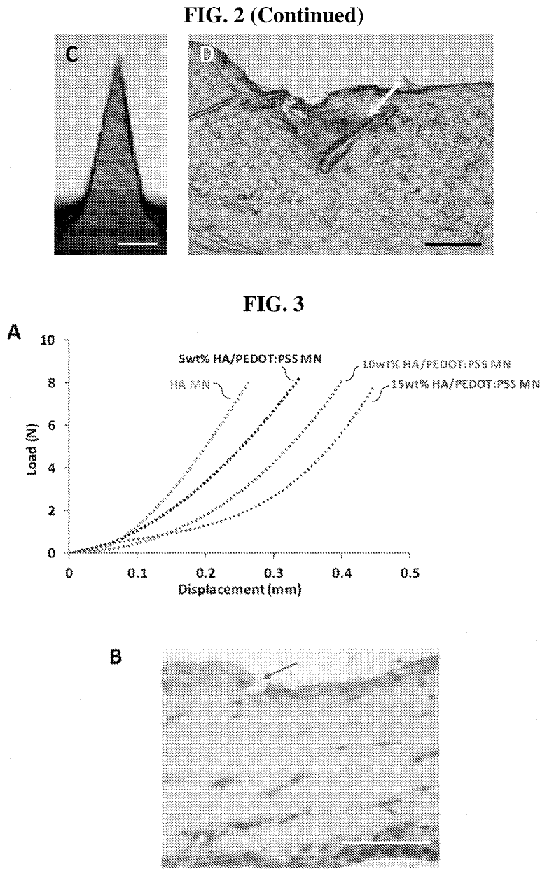

As already mentioned above, the present conductive MNs were fabricated using a conductive polymer and a biodegradable polymer. For accelerated drug diffusion, the conductive MNs were used with iontophoresis. The conductive MN is made up of a mixture of dissolvable polymer (e.g. HA) and conductive polymer (e.g. PEDOT:PSS) (FIG. 2A). The drugs can be mixed with the polymer mixture during fabrication of the device. When the MN patch is applied onto skin, the MN tips are left inside the epidermis layer due to dissolution of the HA polymer matrix that forms the MN tip and base, and contact with interstitial fluid after skin penetration renders dissolution of the MNs instantly, or almost instantly, to release drug molecules encapsulated in the MNs matrix (FIG. 2D). The presence of conductive polymer reduces resistance of skin tissue (e.g. stratum corneum or mucous layer) to enable stronger flow of charges.

To demonstrate synergistic effect of the present conductive MNs and iontophoresis, the MNs are encapsulated with the near-infrared Cy5 dye as a model drug (FIG. 2C) and tested both in vitro and in vivo. These are discussed in the examples further below.

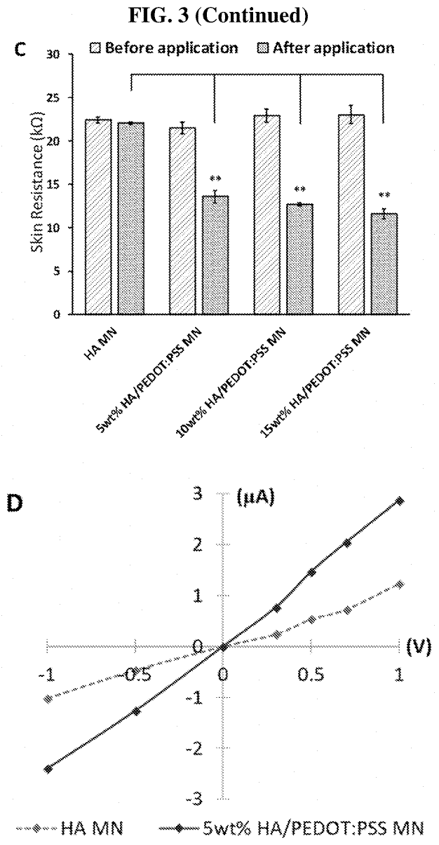

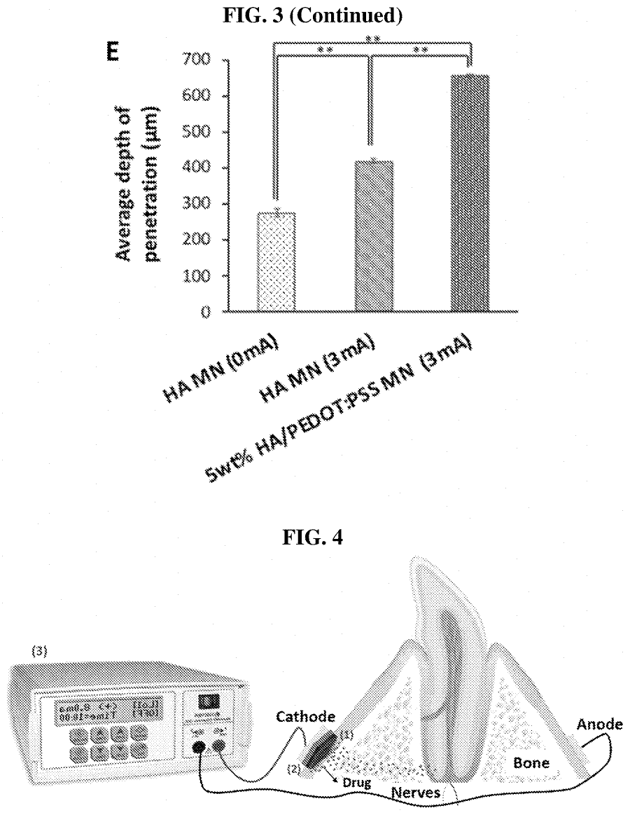

The mechanical strength of HA/PEDOT:PSS MN arrays was studied through an axial compression test. MN patches with different loading concentrations of PEDOT:PSS (e.g. 0 wt %, 5 wt %, 10 wt % and 15 wt %) showed similar load versus displacement profiles (FIG. 3A). The 10×10 PS MN arrays were able to withstand an axial load of 8N without fracture and this corresponds to an axial load of 0.08 N per needle. This indicates that the mechanical strength of the MN array is influenced by the presence of PEDOT:PSS (FIG. 3A). The higher the concentration of PEDOT:PSS, the greater displacement observed. In other words, the MN became softer with PEDOT:PSS, and MNs containing 5 wt % PEDOT:PSS were selected for further demonstration.

The actual skin insertion capability of the HA/PEDOT:PSS MN was examined using fresh porcine cadaver skin. A 5 wt % HA/PEDOT:PSS patch was used as a non-limiting example for demonstration. Using a thumb to press was sufficient for the MNs to penetrate the skin tissue. Subsequent histology study revealed the successful penetration of the MN through the epidermis layer (FIG. 3B). Next, conductivity of the MN array was evaluated by measuring the resistance of mice skin before and after insertion of the MN using a multimeter. The skin resistance was measured immediately after insertion of the MN patches. From the results obtained, it is observable that PEDOT:PSS on the epidermis layer of skin tissue significantly decreases resistance of the skin (FIG. 3C). The electrical conductivity of HA MN and HA/PEDOT:PSS arrays were also calculated using the representative I-V curves at −1 to 1 V bias (FIG. 3D). Using the slope of the linear regression in the ohmic region, the results showed that HA/PEDOT:PSS MN array advantageously has at least 3-fold higher conductivity than HA MN array.

FIG. 3E shows the results where combination of the present conductive HA/PEDOT:PSS MN and iontophoresis produces a 1.5-fold increase in the dye penetration depth compared to a treatment group using non-conductive MN and iontophoresis.

### Example 1C: General Demonstration of Iontophoresis and Dissolvable Conductive HA/PEDOT:PSS MN Array

Briefly, the following steps are performed (FIG. 4).

The component labeled as (1) is the conductive MN patch (1 cm×1 cm) which is first applied on the buccal oral mucosa. The component labeled (2) is a commercially available anaesthetic gel that is applied on the top of the inserted MN array. The component labeled (3) is the iontophoresis unit with electrodes (cathode and anode) that are connected to top of the inserted MN array and the lingual mucosa side of the tooth to be anaesthetized, respectively. Using the iontophoresis unit, a low-voltage current is applied that drives the anaesthetic molecules from the drug reservoir through the micron-sized holes in the oral mucosa and into the alveolar bone targeting the nerves at the tooth apices to render a numbing effect.

The present conductive dissolvable MNs have been investigated for transdermal drug delivery and shown to penetrate skin to create transient aqueous conduits for drug molecules to permeate through. Nevertheless, drug flow within the tissue may be solely dependent on passive diffusion leading to slow drug onset. To accelerate the MN-mediated drug diffusion process, the combination of using both MNs and iontophoresis has been demonstrated herein.

Iontophoresis uses a low-voltage current to drive and enhance delivery of charged and/or uncharged drug molecules across intact skin through electro-repulsion and electro-osmosis. MNs have been used in pre-treatment of skin to create micron-sized holes to allow drug molecules to permeate into the tissue. Iontophoresis is then applied to drive the molecules from the skin surface further into the tissue for quicker systemic effect. In the present disclosure, it has been shown that use of the present conductive MNs not only create micron-sized holes for drug penetration but also significantly reduce resistance of, for example, the oral mucosa. A synergistic enhancement in drug permeation is observed when such a combination is employed. The reduced mucosa resistance enables a greater flow of drug molecules within the oral mucosa when iontophoresis is applied. This allows drug molecules to be driven rapidly from the surface of the oral mucosa into the bone tissue to render an anaesthetic effect. As the present MNs are capable for use in anaesthetic delivery, the pain, fear and/or anxiety associated with needle injections are thus eliminated. The micron-scale needles specifically penetrate the superficial epithelial layer of the oral mucosa without contacting underlying nerve endings in the deeper lamina propria.

In addition, the present MNs are patient-friendly in that the MNs eliminate the phobia associated with needle appearance. Patient acceptance of this technology leads to greater efficiency of, for example, dental practice as time is not wasted in calming patients during dental treatments, thereby forging better relationship between the dentist and patient for smooth delivery of dental procedures. The reduction of dental anxiety also leads to non-avoidance and less rescheduling of dental appointments, thus improving business operations and finances.

### Example 1D: In Vitro Transdermal Drug Delivery with Dissolvable Conductive MNs

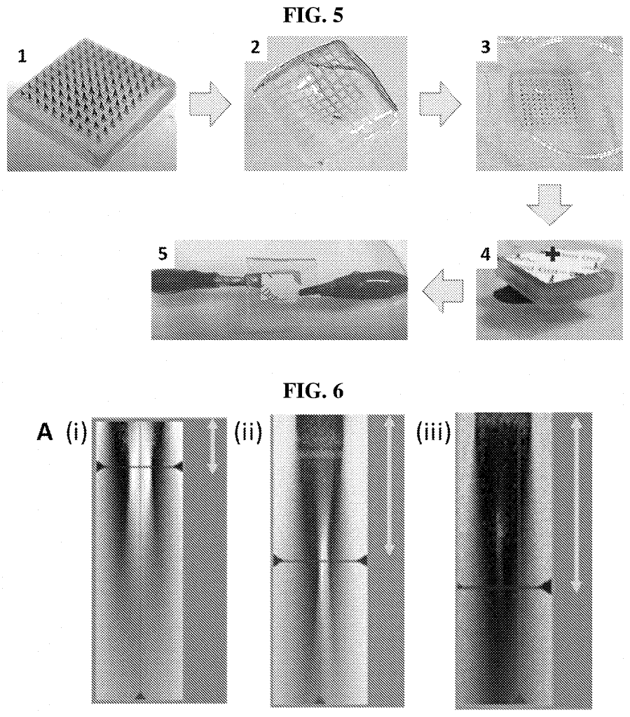

To investigate the potential of conductive MNs for transdermal drug delivery in combination with iontophoresis, a 1.4 wt % agarose gel was used as an in vitro model replica of skin with similar water content and integrity (FIG. 5). There are three steps in the procedure, namely:

(1) The MN patch was applied onto the agarose with a thumb force for 30 seconds and the base layer was then removed.

(2) Cathode electrode was placed on site of application of MN whilst the anode electrode was placed directly at the bottom of the agarose gel.

(3) A low voltage current (3 mA/cm²) was applied for 3 minutes.

As a proof-of-concept, a positively charged fluorescent dye Cy5 was delivered into the agarose gel.

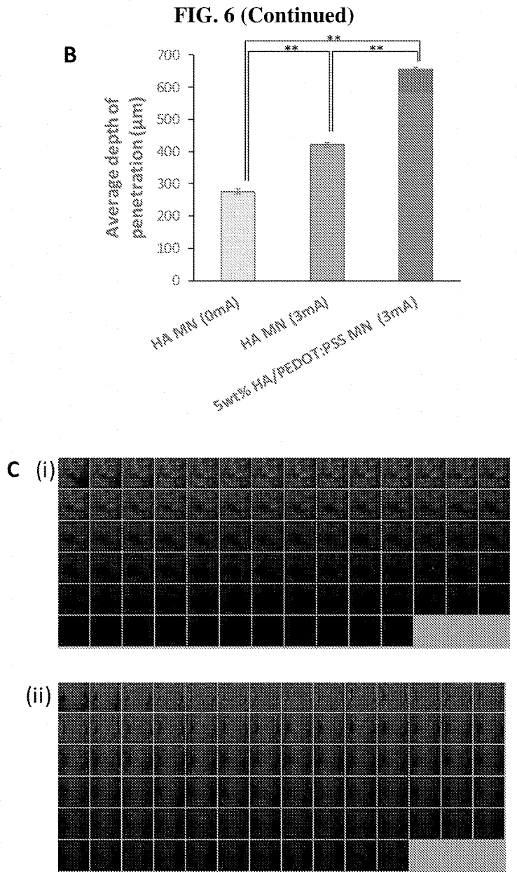

The depth of Cy5 dye penetration in 1.4 wt % agarose was observed using Confocal Scanning Laser Microscopy (CSLM). It is observed that the use of non-conductive HA with iontophoresis with MN resulted in a 1.5-fold increase in the penetration depth, whilst a combination of conductive HA/PEDOT:PSS MN and iontophoresis resulted in a 3-fold increase in the penetration depth (FIGS. 6A and 6B). Images of the treated agarose in the z-axis plane displayed a significantly deeper penetration when conductive MN was used in conjunction with iontophoresis, indicating synergistic effect of such combined use (FIG. 6C(ii)).

### Example 1E: In Vivo Transdermal Drug Delivery with Dissolvable Conductive MNs

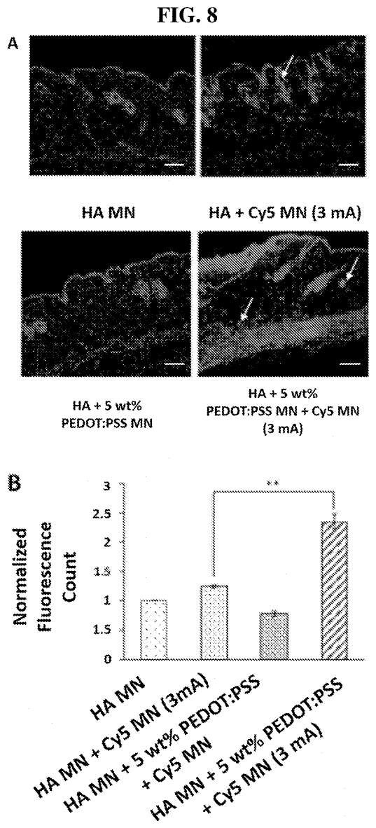

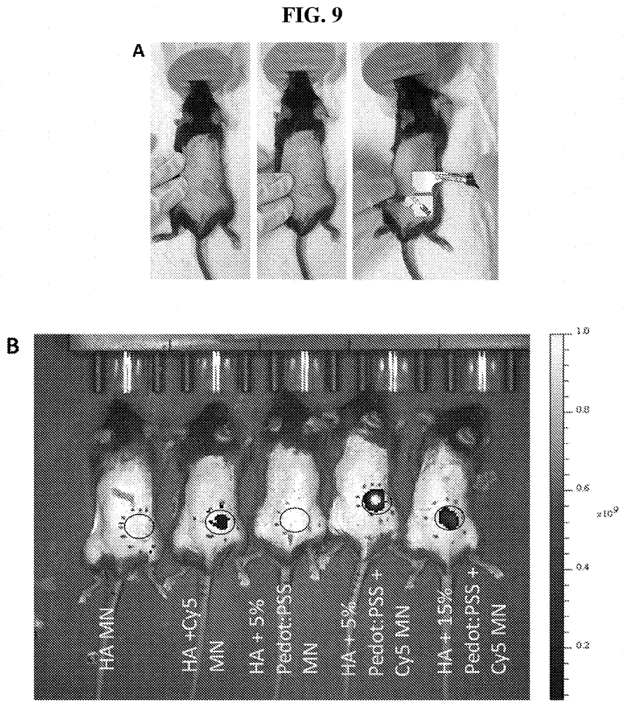

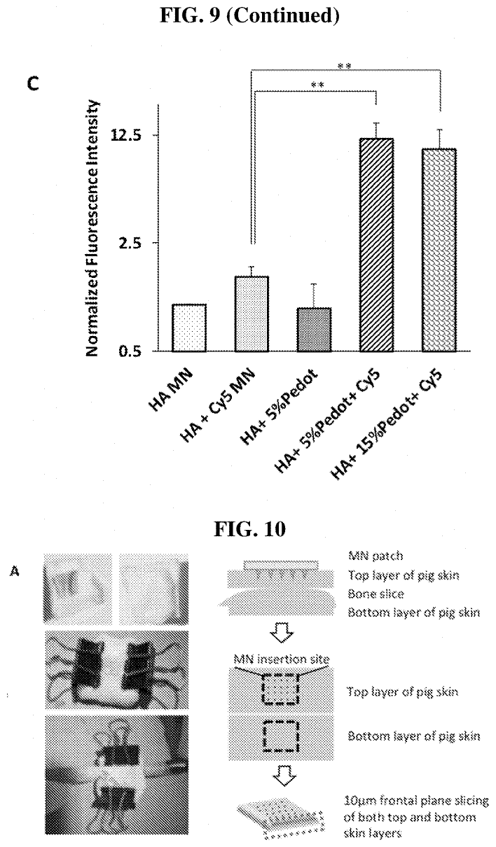

The effectiveness of transdermal drug delivery was demonstrated through an in vivo approach using the mice models (FIG. 7). Briefly, the Cy5 loaded MN patch was firmly pressed into the dry dorsal skin of C57/Bl6 mouse back. After the removal of the MN base patch, both the anode and cathode electrodes were placed side by side with cathode electrode placed in situ of MN application site. Similarly, a low voltage current with current flux density of 3 mA/cm² was supplied for 3 mins. The extent of Cy5 dye penetration in this model was measured and quantified through the In Vivo Imaging System (IVIS) (FIG. 9B) and histology of the treated skin (FIGS. 8A and 8B).

When there was no Cy5 in the MN (labeled as HA MN and HA+5% Pedot:PSS), there was no fluorescence signal on mice skin (FIGS. 9B and 9C). The incorporation of Cy5 into HA MN slightly increased the fluorescence signal. The biggest increase was observed for the HA+5% Pedot:PSS MN and HA+15% Pedot:PSS MN with Cy5 (10-fold enhancement in the IVIS signal). This demonstrated the influence of using a conductive MN with iontophoresis to enhance drug permeation in skin. It is also notable that the skin of the treated mice was cleanly wiped off before the IVIS measurement was performed to ensure no discrepancies from any remaining Cy5 on the skin surface.

C57/Bl6 mice was also used as an in vivo model to evaluate depth of Cy5 penetration across mice skin tissue. The extent of dye penetration was measured and quantified through the In Vivo Imaging System (IVIS) and histology studies. The treated skin was surgically removed for histology studies. Skin sections of 10 μm were obtained using cryosections and stained with Hoechst to differentiate different skin layers, and to visualize and compare the depth of dye penetration.

On mouse skin treated with HA MN and iontophoresis, there was a small amount of Cy5 signal on stratum corneum/epidermis layers (FIG. 8A). Mice skin treated with the conductive HA/PEDOT:PSS MN and iontophoresis showed much stronger Cy5 signal (2 times) (FIG. 8B). More excitingly, Cy5 signal was seen the deeper layer of skin, suggesting the improved penetration of Cy5 by using the conductive MNs and iontophoresis. In other words, in comparison to mice skin treated with a non-conductive MN and iontophoresis, fluorescence particle count was 2-fold higher in the stratum corneum/epidermis and dermis layer in mice skin treated with the conductive MN and iontophoresis, suggesting the establishment of a deeper penetration effect from the conductive MN patches (FIGS. 8A and 8B).

### Example 1F: Ex Vivo Study on Drug Delivery Through Bone Penetration with Dissolvable Conductive MNs

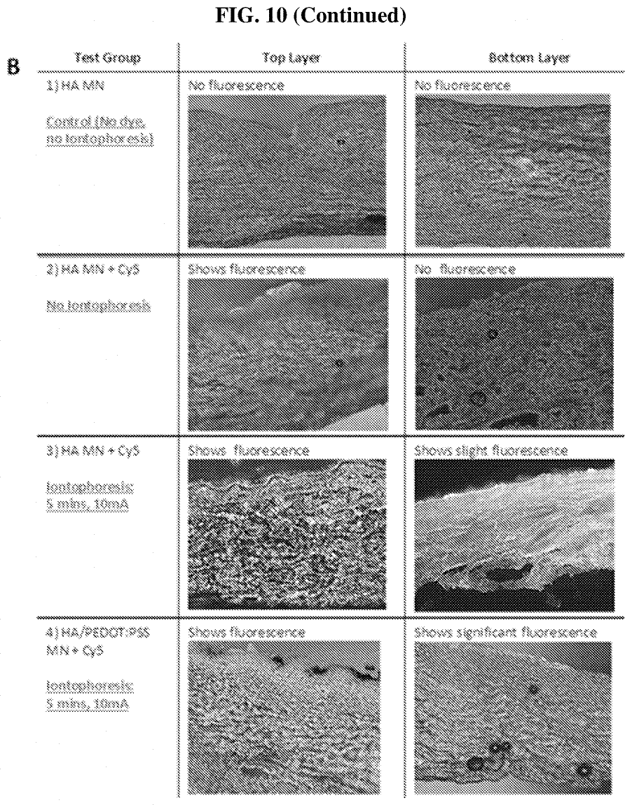

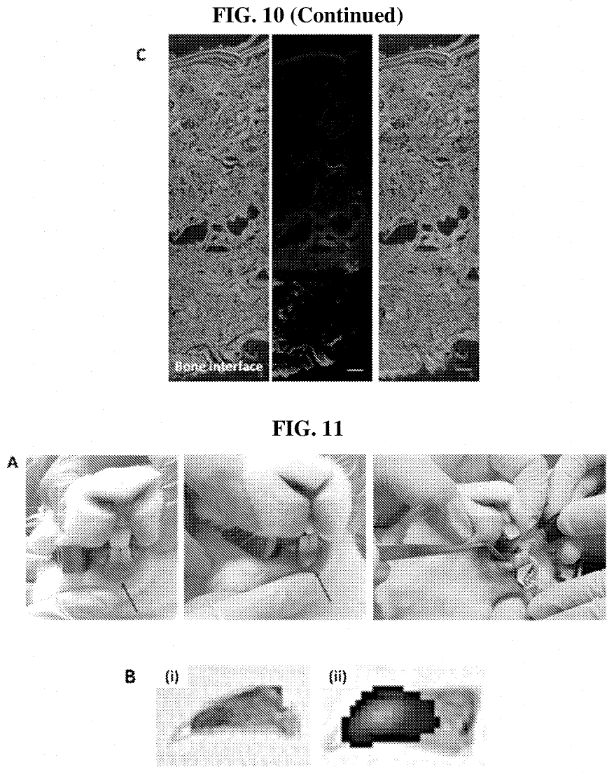

The synergistic effects of combined use of conductive MN array and iontophoresis have been demonstrated to accelerate drug penetration effect through in vitro and in vivo models. To translate the use of the present conductive MN for anaesthetic delivery in oral tissues, it is crucial to determine if drug permeation through the alveolar bone is possible. Briefly, an ex vivo study was conducted using a phantom model consisting of sections of rabbit mandible bone and pig ear skin (FIG. 10A). Following treatment, the top and bottom layer of the pig skin were taken apart from the bone for histology studies to evaluate the depth of dye penetration. For skin treated with a combination of non-conductive MN and iontophoresis, distribution of dye particles was mostly observed in the stratum corneum layer to the dermis layer of the top layer of the skin suggesting drug penetration through the bone was not achieved (FIG. 10B). Advantageously, for skin treated with conductive MN and iontophoresis, a greater proportion of the dye particles was observed in the bottom layer. Notably, a stronger fluorescence signal was observed at the skin-bone interface portion of the bottom layer of skin (FIG. 10C). In summary, the results demonstrated the advantage of using the present conductive MN and iontophoresis to achieve deeper drug penetration capable of surpassing the alveolar bone.

In detail, rabbit mandible bone (1 cm×1 cm) was sandwiched in between 2 layers of pig ear skin (2 cm×1.5 cm) and secured using paper clips. Such a phantom model was designed to study the possibility of dye penetration through bone samples (FIG. 10A). The respective MN patches were applied onto the epidermis of the top layer of the for 1 min before performing iontophoresis (if applicable). Both the top and bottom pig skin layers were then removed apart immediately after treatment and detached from the bone sample.

The portion of the skin treated with MN was cut out and frozen before cryosectioning was performed to obtain 10 μm slices of tissues. Confocal microscopy images (FIG. 10B) showed presence of Cy5 dye in the bottom layer of the skin for samples treated with iontophoresis, suggesting that the use of iontophoresis facilitated the movement of the dye across bone. However, the skin samples treated with the conductive HA/PEDOT:PSS MN showed greater penetration of Cy5 dye across the bone sample onto the bottom pig skin layer. This helps to demonstrate that whilst iontophoresis is sufficient to achieve dye penetration through the bone sample, it is crucial that the present conductive MN is used in the treatment to produce a deeper penetration for specifically and effectively targeting the nerves that lie beneath the bone sample in actual application.

### Example 1G: In Vivo Efficacy Study of Dissolvable Conductive MNs Using Rabbit Model

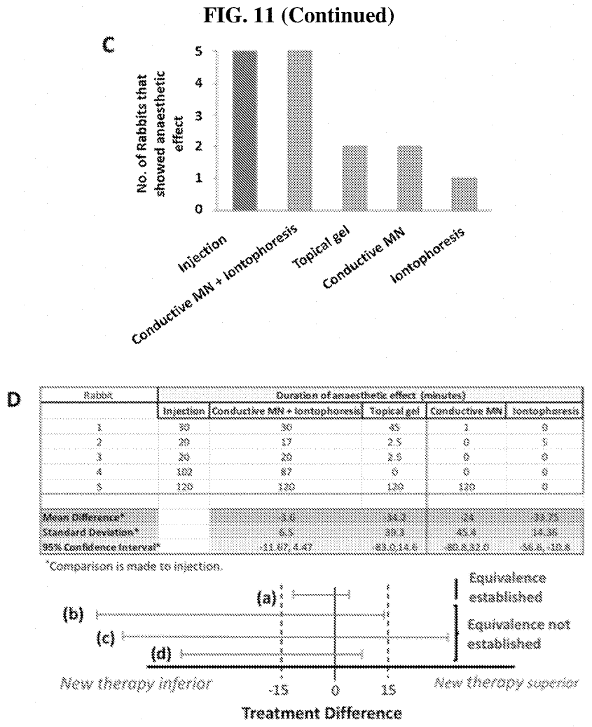

Using the present dissolvable conductive MN, the efficacy of lidocaine delivery for achieving local anaesthesia was studied in a clinically relevant rabbit incisor model (FIG. 11A). The current magnitude and duration of iontophoresis application was pre-determined using ex-vivo bone samples from rabbits. Using IVIS, strong fluorescence signals in the bone samples was observed for 3 mA and 4 minutes, indicating most of the dye had penetrated into the bone (FIG. 11B). The rabbits were subjected to five different local anaesthetic delivery procedures, which were injection using conventional needle and syringe, topical gel application, the present conductive MN alone, iontophoresis alone, and a combination of the present conductive MN and iontophoresis. The pain threshold of the rabbits prior to and after treatment was tested by applying a pain stimulus using an electric pulp tester. All 5 rabbits achieved dental anaesthesia (i.e. no response to pain stimulus applied to the tooth) when treated with conductive MN and iontophoresis (FIG. 11C). The time taken for onset of anaesthetic effect was immediate (at 0 min time point) when the rabbits were treated with either needle injections or conductive MN and iontophoresis. Using a margin of equivalence of 15 minutes at a 95% confidence interval for duration of action, the anaesthetic effect outcome achieved with the present technology is equivalent to the current gold standard of using needle and syringe injection (FIG. 11D).

In summary, the dissolvable and conductive MN array when used in combination with iontophoresis demonstrated its ability to provide quick and deep drug penetration through oral mucosa and bone to reach the nerves supplying sensation to teeth enabling an efficacious and painless delivery method for dental anaesthesia, as one of the many examples of applications.

### Example 2A: Swellable Conductive HA/PEDOT:PSS MN Array

The above examples showed that the doping of PEDOT:PSS polymeric particles within the matrix of HA can result in a conductive polymeric MN array. The use of HA and PEDOT:PSS in the fabrication of a conductive MN results in a dissolvable conductive MN array that disintegrates, or immediately disintegrates, upon insertion into the oral mucosa. Apart from such a dissolvable MN array, the present disclosure also provides for use of MeHA and PEDOT:PSS to form a conductive swellable MN array that attains a swollen morphology within the oral mucosa.

Advantageously, the above examples already demonstrate that the use of a dissolving conductive MN is effective in the delivery of anaesthetic molecules to the rabbit incisors. The efficacy of drug delivery using a dissolving conductive MN in parallel with iontophoresis was determined to be equivalent to the traditional method of needle and syringe delivery at a 95% confidence interval. Further advantageously, the use of swellable conductive MN in combination with iontophoresis results in a stronger electro-osmotic flow that provides a quicker migration of drug molecules from the site of insertion into the nerves residing within the alveolar bone.

Briefly, HA is functionalized with methacrylate to obtain methacrylate-crosslinked HA (MeHA) that can be further crosslinked via a free radical polymerization under ultraviolet (UV) illumination. To fabricate swellable conductive MNs, MeHA polymer of concentrations, e.g. 25 mg/ml to 100 mg/ml, can be doped with PEDOT:PSS polymer in varying weighted concentrations of 5 to 20 wt %. PEDOT:PSS polymer is mixed under constant stirring (300 to 500 rpm) with MeHA and a photoinitiator for 2 days to obtain a homogeneous solution. This solution is then added to plasma treated PDMS mold and centrifuged before drying for 3 days. The MNs are then crosslinked using UV illumination for a time duration of 5 minutes to 15 minutes.

Further details on fabrication of the swellable conductive MN array is discussed below.

### Example 2B: Fabrication of Swellable Conductive MeHA/PEDOT:PSS MN Array

Synthesis of a HA polymer solution has already been described in the above examples and shall not be iterated for brevity.

To fabricate a crosslinked MeHA MN patch, MeHA (50 mg/mL) and a photoinitiator (Irgacure 2959, 0.5 mg) were dissolved in DI water. The mixture was casted into the plasma-treated PDMS mold until filling up the cavities. Then, the PDMS mold was centrifuged at 4000 rpm for 3 mins to force material to fill up any needle voids. Additional mixture solution was added to produce a robust backing. After drying at room temperature in a fume hood (about 12 hrs), MeHA-MN patches were carefully separated from the mold and trimmed, and then were exposed to UV light (wavelength=360 nm, intensity=17.0 mW cm⁻², model 30, OAI) for a period of time (3 mins, 5 mins, 10 mins, 15 mins, etc.).

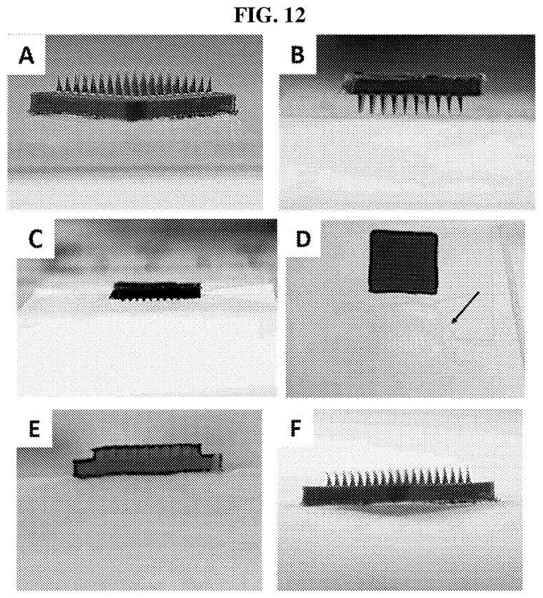

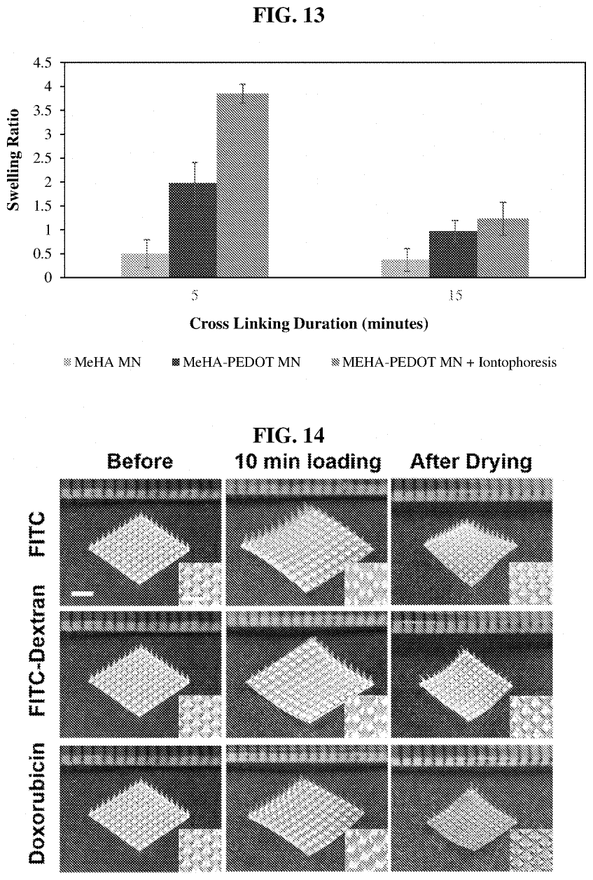

The swelling effect of the conductive MeHA-PEDOT microneedle array under the influence of a low-voltage current is investigated. The MNs (with different degree of crosslinking) were weighed before insertion into a parafilm covered 1.4 wt % agarose gel (FIG. 12A to 12F). After 1 minute of incubation, the MNs were removed carefully and quickly weighed. Using the swelling ratio obtained, the extent of cross-linking degree on the swellability of the microneedles is determined. The results showed that a shorter crosslinking duration contributed to a greater swelling effect and more fluid was absorbed into the MN matrix. When a conductive MN was combined with subsequent iontophoresis treatment, the MNs swelling ratio increased 2-fold. The ability of the present conductive MN (MeHA-PEDOT) to swell more in comparison to the non-conductive MN (MeHA) allows it to further contribute to the electro-osmosis effect when iontophoresis treatment is performed. FIG. 13 shows the swelling behaviors of crosslinked MeHA-MN patches studied in 1.4 wt % agarose gel after 1 minute of incubation.

### Example 2C: Drug Loading in Swellable Conductive MN Array

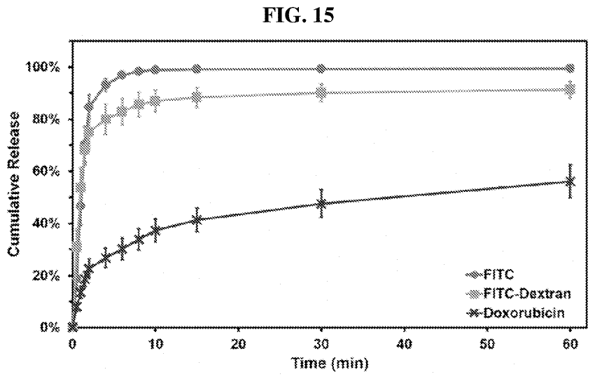

The drug loading capabilities of MeHA MNs was investigated by using model drugs of different molecular weights, mainly fluorescein isothiocyanate (FITC), FITC-Dextran and doxorubicin hydrochloride (Dox). The innate ability of the MeHA MN arrays to absorb fluid is taken advantage of to load the drug molecules into the MN tips. All three drugs are dissolved in PBS which were then used to equilibrate MeHA MN patches. After 10 minutes of incubation with the drug solution, the MNs were left to dry in the fumehood (FIG. 14). The amount of drug encapsulated in the MN was determined by immersing the drug loaded MeHA in 1×PBS. By obtaining a 50 μL of the solution at various time points, the fluorescence intensity was measured to determine the drug release profile of the three different drug molecules. As seen from FIG. 15, the molecular weight of the drugs affects the drug loading capability through the swelling effect. FITC and FITC-Dextran with low molecular weights (3 to 5 kDa) were easily loaded into the MN tips as compared to bigger Dox molecules (20 kDa).

Next, the release profiles of drugs from the MeHA MN patches were examined. As depicted in FIG. 3B, more than 80% of FITC and FITC-Dextran were released from the MN patches within 30 minutes. 50% release from Dox-loaded MN patches was observed in the same period.

### Example 2D: Advantages of the Swellable Conductive MNs Array

The pores created by the dissolvable conductive MNs in the mucosa close almost immediately following MN dissolution. This is suitable for applications that do not require the pores to remain open for a long period of time.

Meanwhile, the swellable and non-dissolvable conductive MN array using hydrogel-forming polymers to develop MNs with the ability to swell when inserted into the oral mucosa, when used in combination with iontophoresis, results in an electro-osmotic effect that further increases the iontophoretic flux and hence promotes quicker drug delivery into the bone tissue from the swelling effect. The swellable MN configuration provides the prospect of having a continuous drug flow into the tissues when a current is applied resulting in a more efficient drug delivery platform. In addition, the swellable and non-dissolvable conductive MNs can be removed completely after its use, limiting the residue left inside soft tissues.

### Example 2E: Further Discussion—Procedures for In Vitro Studies Using Rabbit Model

The above examples already demonstrate a highly conductive swellable MN array useable in combination with iontophoresis for accelerated drug delivery. The low molecular weight HA, as a non-limiting example, used in fabrication of the dissolvable MN array is modified to form a hydrogel-forming HA for fabricating the swellable conductive MN array. The swellable conductive MNs swell, for example, upon insertion into oral mucosa and facilitate delivery of drug molecules from an anaesthetic gel that can be applied on top of the MN array (FIG. 24).

The hydrogel-forming MNs aid further acceleration of iontophoteric effects in addition to the iontophoteric effects achieved with dissolvable MNs. This is because application of an electrical current leads to a dramatic increase in the swelling of the hydrogel-forming MN due to the enhanced water uptake by electro-osmosis. This is capable of inducing a 5-fold increase in the MN surface area that greatly facilitates and enhances movement of charged molecules. Hence, the use of covalent crosslinking to incorporate stable internal networks to achieve hydrogel-forming HA MNs provides for a HA polymer that can absorb fluid without dissolving.

### Example 2F: General Discussion—Development and Characterization of Swellable Conductive MN Array

The highly conductive and swellable MN array demonstrated in the above examples is based on a hydrogel-forming HA and PEDOT:PSS. To achieve hydrogel-forming HA, HA polymer modification was done by crosslinking the hydroxyl or carboxyl groups of HA polymer with functional moieties. Various crosslinkers and crosslinking methods (e.g. methacrylate, furan and catechol) have been used. Without being limited to but for the sole purpose of demonstration, methacrylate crosslinkers were discussed in the present disclosure. The reagents and polymers used in the fabrication of the MN array are biocompatible and of medical grade.

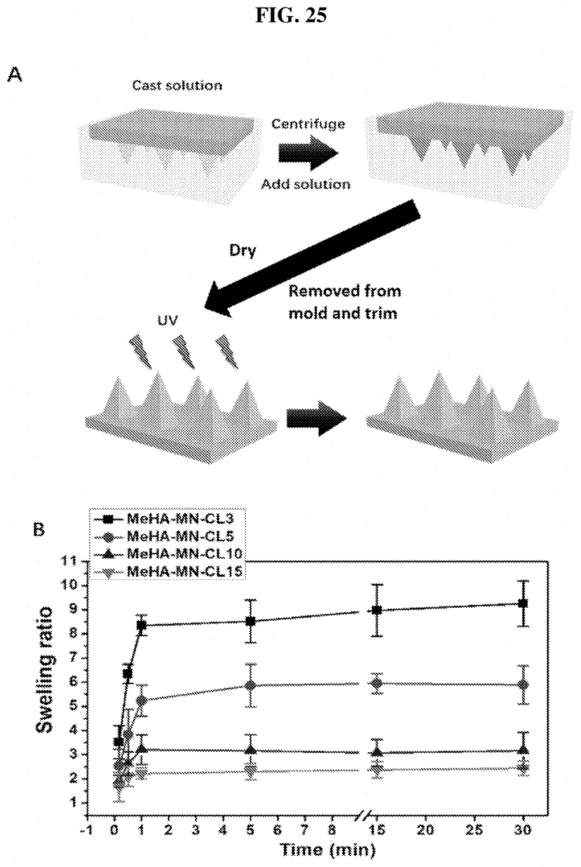

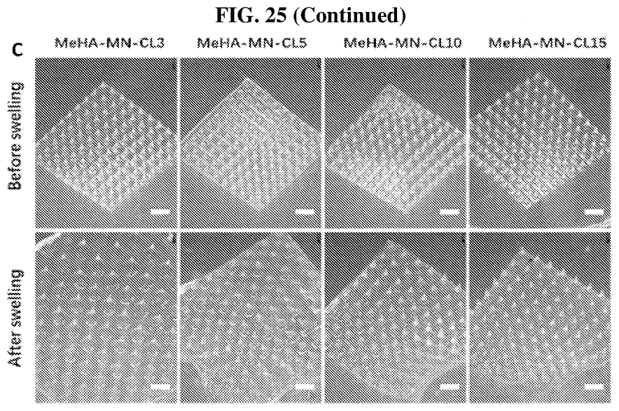

HA modification using methacrylic anhydride followed by further crosslinking via free radical polymerization under UV illumination can be done to result in highly swellable methacrylated hyaluronic acid (MeHA) MN arrays (FIG. 25A). Different UV exposure time resulted in a significantly different swelling profile and morphology of the MNs as shown by the swelling ratios (FIGS. 25B and 25C). This observation indicates the crosslinking degree of MeHA plays a role in the amount of fluid volume MeHA MNs can absorb. As the swellability of the MNs influences the iontophoresis effect, the crosslinking degree can be tuned for maximum swellability. Next, the influence of PEDOT:PSS polymer on the crosslinking degree and UV exposure times can be evaluated to design and fabricate hydrogel-forming HA/PEDOT:PSS MN array that is capable of retaining conductivity while absorbing maximum fluid.

Using the modified HA polymer incorporated with PEDOT:PSS polymer, micromolding techniques are used in the fabrication of the MN array. Briefly, a negative polydimethylsiloxane (PDMS) mold was first made from a designed stainless steel MN template. The modified HA solution which is incorporated with PEDOT:PSS was then casted into the PDMS mold and dried naturally. For the different chemistries discussed above, different steps can be performed for further crosslinking of MNs, e.g. crosslinking of MeHA MNs was conducted with UV exposure after the solution is dry, crosslinking of furan-HA MNs was done during the evaporation step by mixing the crosslinking agents, and the crosslinking of catechol-HA (CA-HA) MNs was achieved by exposing MNs in a solution containing NaIO₄ and NaOH.

Following the different types and durations (i.e. 3, 5, 10 and 15 minutes) of cross-linking, swelling ratio of the respective MNs was obtained by measuring the change in mass of the MN patch after immersion into phsophate buffer saline (PBS) for 1, 3 and 5 minutes. Optical coherence tomography imaging was used to monitor the in situ and real-time swelling behaviours of the MNs for comparing the swelling speed of different crosslinked HA MNs. Characterization of the different MN designs was also done to ensure that the MNs are suitable for oral mucosa penetration. The mechanical properties are examined with Instron 5543 Tensile meter to obtain load versus displacement profiles. In addition, penetration efficiency of MNs were also tested by pressing MNs into freshly harvested rabbit buccal mucosa and histology studies were performed to evaluate the depth of penetration. Next, conductivity of the swellable MNs were studied by performing transcutaneous electrical resistance measurements using rabbit buccal mucosa mounted over a Franz diffusion cell with the receptor compartment containing PBS (pH 7.4). The electrodes were connected to a multimeter and placed on both side of the tissue portion to measure resistance. MN array are inserted into the mucosa tissue and left within for continuous measurements of resistance.

The swellable conductive MN array was evaluated for its permeability to the applied anaesthetic gel, followed by subsequent drug release profile analysis. In vitro permeation studies were performed using Franz diffusion cells and rabbit buccal mucosa to assess the amount of lidocaine delivered under varying iontophoretic flux. Rabbit buccal mucosa tissue is cut to the size of the diffusion cell area and the pieces are sandwiched between donor and receptor cells. The MN arrays can be inserted into the centre of the tissue section using a thumb press followed by application of a commercially available anaesthetic gel. The active electrode can be placed directly on top of the gel layer and MN array, whilst the inactive electrode can be placed onto the tissue placed on the receptor compartment of the Franz cell. At predetermined intervals, aliquots from the receptor can be collected and replenished with fresh receptor solution. The samples can be analysed using high-performance liquid chromatography (HPLC) techniques to detect the amount of lidocaine released through the mucosa tissue. These studies were done to deduce the drug loading concentration necessary to derive the anaesthetic dose required for local anaesthesia.

In addition, the iontophoresis conditions can be tuned and parameters such as voltage and duration of current applied can be evaluated to achieve desired drug dosage. The depth of drug penetration were studied using 1.4 wt % agarose and rabbit buccal mucosa. In such a study, a charged, fluorescent dye can be used as a model drug and delivered using the conductive MN and iontophoresis. Following treatment, the agarose/tissue samples can be imaged using confocal microscopy to observe the penetration depth of dye molecules. Histology studies can be performed for the mucosa tissue samples followed by imaging to observe the depth of penetration. Any visible signs of inflammation can also be monitored.

### Example 2G: General Discussion—Procedures for In Vitro Studies Using Rabbit Model

The delivery of anaesthetic agents, using the swellable MN patch (or the dissolvable MN patch) and iontophoresis system, through mucosal tissue and alveolar bone was also examined using harvested rabbit jaws. Rabbit carcasses were obtainable from the Singhealth Experimental Medicine Centre. For each carcass, 2 sites per dental quadrant were used (i.e. the first premolar and second molar sites where the bone thickness differs). Anaesthetic-incorporated hydrogel have to be carefully applied onto one end of the microneedle patch and empty (no drug) hydrogel have to be applied to the other end. The patch can be pressed on the buccal and lingual mucosal tissue around the tooth of interest. Iontophoresis can then be performed where a current can be passed from the cathode to anode. The teeth and surrounding dentoalveolar tissue can be sampled for quantifying the fluorescent anaesthetics delivered to the mucosa and alveolar bone tissue surrounding the root apices. Different voltages can be used to analyze the relationship between the voltage, and the rate and depth of drug penetration.

### Example 2H: General Discussion—In Vivo Evaluation for Efficacy and Efficiency Using Dental Pain Model of Rabbits

For proof of concept, an in vivo pilot study was carried out on live rabbit dental models. Quantitative measurements of the onset and duration of local anaesthesia can be investigated.

Briefly, the rabbit is to be lightly sedated using Acepromazine at 1-2 mg/kg injected intramuscularly. Acepromazine is used to result in a sedative effect without analgesia. It facilitates placement of the MNs and iontophoresis electrodes and still allow pain assessment. For each animal, 1 site per rabbit is used (i.e. the bottom incisors). In the treatment group, the MN patch is gently pressed onto the buccal mucosal tissue beneath the tooth and a commercially available anaesthetic gel is applied onto the MN array. Iontophoresis can then be performed with varioous conditions of current and application duration. In the control group, anaesthetic can be delivered by local infiltration using a conventional dental syringe, needle and local anaesthetic cartridge. For evaluation of dental anaesthesia onset and degree of anaesthetic effect, the method is described as follows.

A voltage is applied to the rabbit's tooth using an electric pulp tester until the pain inducing threshold voltage is determined. This pain inducing threshold voltage is determined using the rabbit grimace scale by looking out for signs such as orbital tightening, change in nostril shape and licking movements. The pain threshold of the rabbits are recorded before intervention. After application of treatment procedure, the pain threshold to a voltage stimulus are monitored at different time points, e.g. 0 min, 0.5 min, 1 min, 1.5 min, 2 min, 2.5 min, 3 min, 3.5 min, 4 min, 4.5 min, 5 min, 10 min, 20 min, 30 min, 45 min, 60 min, 75 min, 90 min, 105 min, and 120 min. Quantitative measurements of the time taken for anaesthetic onset and duration of anaesthetic effect can be used to determine efficacy of the device. The vital signs (ECG, respiratory rate, heart rate) are also to be monitored during this time. Adverse events can be tracked over 3 days before the animals are euthanized and the dentoalveolus harvested for histology analysis. Signs of necrosis, inflammation or any other abnormalities of the soft and hard tissues can be evaluated in the treated samples.

### Example 3A: Further Examples—Fabrication of Template for Forming the MNs and Fabrication of a MN Patch Using the Template

The master template was prepared using tilted-rotated photolithography approach. Briefly, a thick layer of SU-8 (an epoxy-based negative photoresist) was coated on the surface of an anti-reflective silicon wafer. Next, a mask with pre-designed squares and spacing was placed over the SU-8, which determines the microneedle base diameter and interval, respectively. Afterwards, the SU-8 complex was selectively exposed to UV light at the incidence angle of 18 to 25°, ultimately determining the height of microneedles. The wafer was then clockwisely rotated 90° and the exposure was performed again. A total of four exposures led to master templates with square-pyramidal base structure developed. All microneedles have 150 μm to 200 μm base diameter and 5 μm to 10 μm in tip diameter. The spacing between each microneedle was 1 mm to avoid a high concentration of MNs formed at a localized area (i.e. avoid “a bed of nails”). The patch had a size of 12 mm×12 mm. PDMS mold can then be fabricated by traditional elastomer curing process based on the master templates.

The PDMS mold was coated with a conductive polymer solution before it is centrifuged. The conductive polymer is a conductive polymer with sufficient mechanical strength for penetration into the skin of a subject and can be selected from polypyrrole based polymers, a structure of which is shown below, wherein n may be from 1 to 100,000.

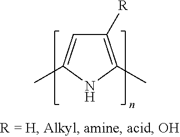

After centrifugation, the excessive solution on the PDMS mold was removed by filter paper. The solvent was then dried to form the microneedles. Subsequently, another solution containing the second type of conductive polymer was added onto the mold to form the base/substrate of the patch.

Hardness and elastic modulus of the microneedles were evaluated with a universal test machine. Morphology of the microneedles was examined with scanning electron microscope (SEM), and the three-point bend test and fatigue fracture test were carried out for examining flexibility of the base.

### Example 3B: Further Examples—Microneedle Patch Prepared with PEG-DA Through Photolithography and Mechanical Characterization

A microneedle patch was prepared through a photo-polymerization method with poly(ethylene glycol) diacrylate (PEGDA) and 2-hydroxy-2-methyl-propiophenone (HMP) (FIGS. 18A and 18B).

Fabrication of a supporting substrate is shown in FIG. 18A. A pre-polymer solution was prepared by mixing PEGDA and HMP. A glass slide was used as a substrate to support two untreated coverslips to form a gap. Another coverslip was aligned on this gap to create a narrow cavity. Pre-polymer solution was introduced and spread into the cavity by capillary force. Then, the set-up was placed under ultraviolet (UV) light source, followed by UV irradiation. Subsequently, the formed supporting patch was removed from glass substrate and dried in an oven (50° C.)

Fabrication of the microneedles is shown in FIG. 18B. Glass slides with thickness of 1 mm were placed on either side of a broader glass substrate to increase the cavity height. Then, the adhesive patch was reversely aligned on the cavity, followed by introducing prepolymer solution into the cavity through capillary action. A photomask with the designed pattern of microneedle array was precisely placed on top of the adhesive patch. The microneedle array was formed due to photopolymerization under the adhesive patch.

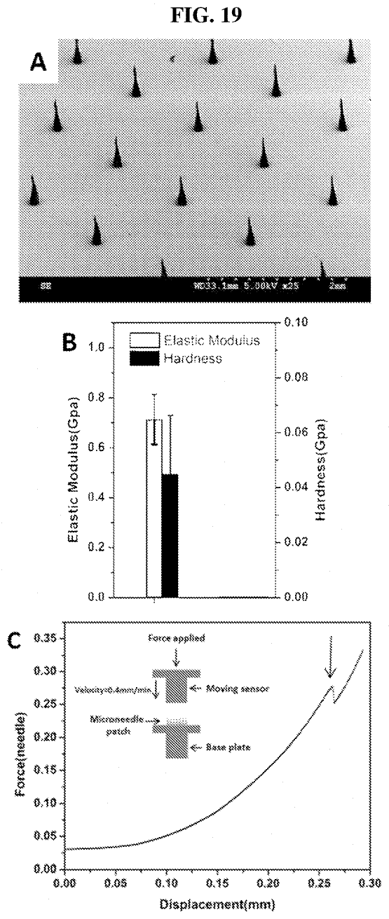

FIG. 19A is a SEM image of a representative microneedle device with a needle density approximately 49 per cm⁻², having a microneedle base diameter of 300 μm, and the microneedle height is 940 μm. All microneedles have a conical shape and are uniformly distributed on the adhesive patch. Hardness and elastic modulus are critical parameters for transdermal devices. As shown in FIG. 19B, the microneedles in the devices have a hardness of about 0.04 GPa and elastic modulus of about 0.6 GPa, which is about 1000 times larger than those of skin layers (elastic modulus: about 0.6 MPa). To penetrate into scars, microneedles need to be strong enough during insertion without mechanical failure. A mechanical compression test was conducted to examine the force required for mechanical fracture of the microneedle device with a universal test machine. As shown in FIG. 19C, microneedles only failed when the compression force reached 0.25 N per needle, which was 5 times larger than the force required for skin penetration (0.05 N per needle).

The microneedle device readily penetrated fresh porcine skin (FIG. 20A). A subsequent histology study revealed that microneedles had penetrated into the dermis layer of porcine skin (FIG. 20B).

### Example 3C: Further Examples—Microneedle Patch Prepared with HA and Fluorescence Dye from Steel Mold

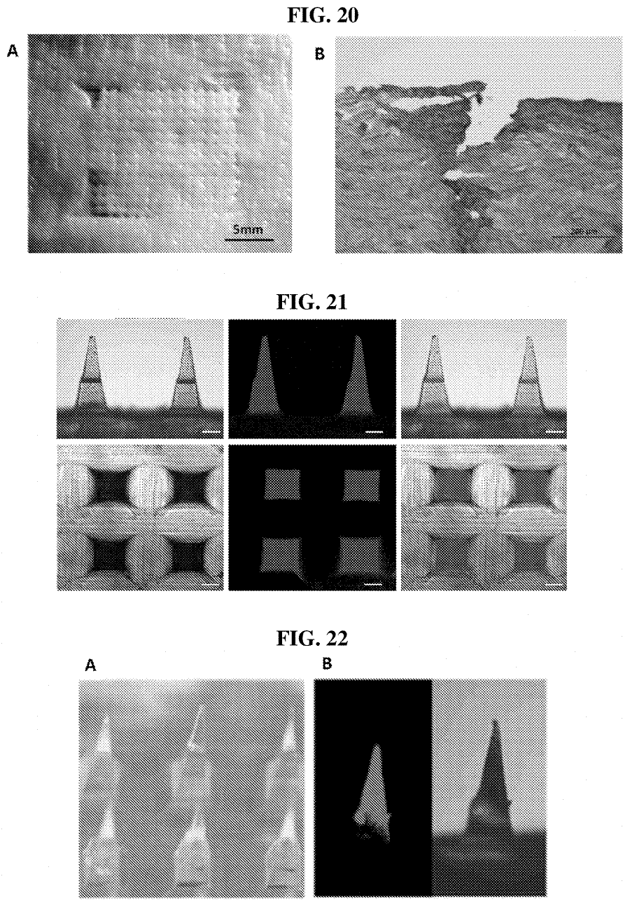

A stainless steel microneedle mold consisting of 100 pyramidal needles (with approximately 600 μm height, 300 μm width at base, 700 μm pitch, and 10×10 array) was created using an electrical discharge machining process. PDMS was prepared by mixing in 10:1 ratio of pre-polymer to a curing agent, and degassed in the vacuum oven for 2 hrs. After placing the stainless steel microneedle in the centre of a petri dish, degassed PDMS was poured over the microneedle mold, and cured for 2 hrs at 70° C. The PDMS reverse-microneedle molds were obtained after the stainless steel microneedle mold detached from PDMS micromold. Then 0.5 g HA containing 1% fluorescence dye was dissolved in 1 ml of distilled water before being added to the surface of the micromold, and centrifuged. Later, blank HA solution was added onto the mold and centrifuged to form the backing layer (base), then dried in room temperature overnight. The dye-loaded MNs patches were peeled off from the micromold. As shown in FIG. 21, the dye-loaded microneedles are fluorescent while the base made with blank HA is non-fluorescent.

### Example 3D: Further Examples—Microneedle Patch Composing HA Base and PLGA Microneedles

With the PDMS mold made above, 200 mg PLGA was dissolved in 1 ml of DMF. 20 μl of the dye-loaded HA solution was added to the micromold, and centrifuged, then dried in the room temperature overnight. A blank HA solution (0.5 g of HA and 1.0 mL of distilled water) was added onto the mold and centrifuged to form the backing layer, then dried in room temperature overnight. The dye-loaded MNs patches were peeling off from the mold. The bright field image of the prepared microneedles is shown in FIG. 22A, which clearly shows the different contrast between the microneedle tip and the base. When the microneedles are visualized under fluorescent microscope (FIG. 22B), only the PLGA tip is fluorescent.

### Example 3E: Further Examples—Conductive Polymer Film and its Electrical Conductivity

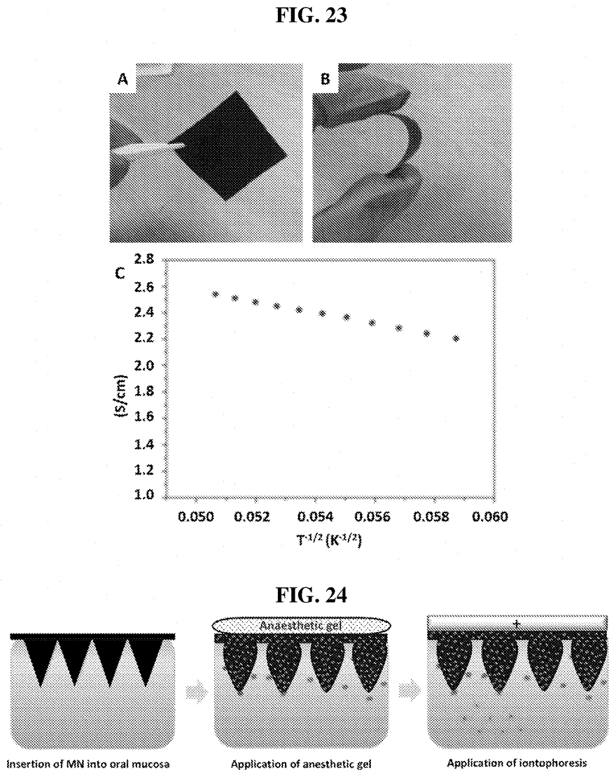

A polypyrrole (PPy) film in the form of a membrane is tested for its suitability as a conductive polymer for incorporation into the various MN arrays described herein. FIG. 23A showed a membrane made with polypyrrole (PPy). This membrane is flexible and mechanically robust (FIG. 23B). The temperature (T) dependence of electrical conductivities (a) is shown in FIG. 23C. The electrical conductivities (a) were about 9.81 S cm⁻¹.

### Example 4: Comparison with Conventional Syringe Approach

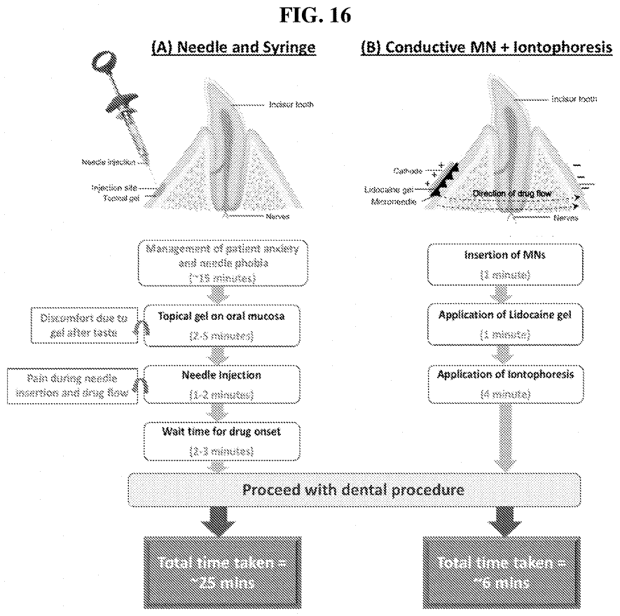

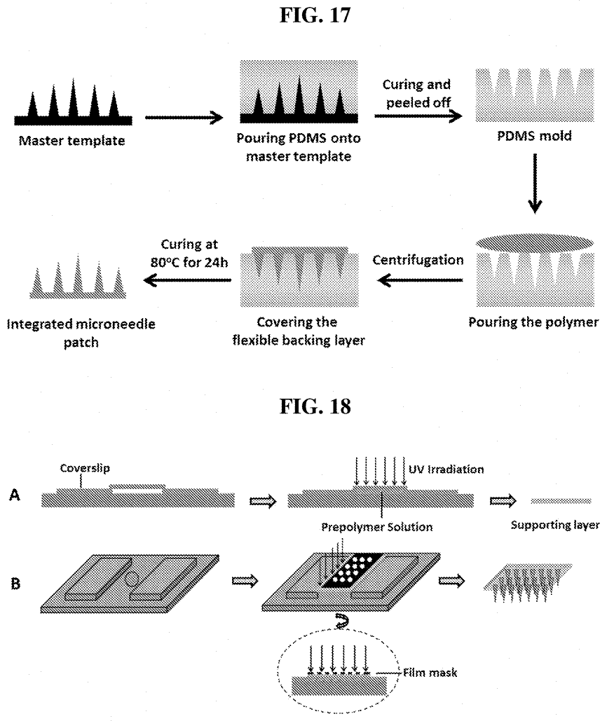

Conventionally, local anaesthetic delivery may be performed in two steps over approximately 10 minutes to achieve anaesthetic effect (FIG. 16—scheme A). Firstly, a topical anaesthetic gel is applied on the buccal and lingual mucosa tissues surrounding the site of treatment. This gel is left on for approximately 2-5 minutes to cause sufficient numbing of the superficial tissues. Next, a needle is penetrated through the muccobuccal tissue to deliver the anaesthetic solution for 1-2 minutes. The anaesthetic solution then diffuses over time through the alveolar bone to reach the sensory nerves surrounding the roots of the teeth which usually takes 2-3 minutes. Patients expressing dental anxiety require a greater level of understanding, good communication and a phased treatment approach which further prolongs the drug administration procedure by at least 15 minutes.

With the present conductive MNs array, there is significant reduction in the time taken for anaesthetic delivery as compared to the traditional needle and syringe method. The present method is patient-friendly and painless, thus eliminating the need to manage patient anxiety and needle phobia which takes up a bulk of dentists' time before dental treatment can take place. In the use of dissolvable conductive MNs and iontophoresis, 3 steps and a total improved time of 6 minutes are required (FIG. 16—scheme B). Firstly, the MNs are pressed on the oral mucosa tissue for approximately 1 minute to ensure proper implantation and complete dissolution. Next, an anaesthetic gel is applied at the site of MN insertion and left on for 1 minute to facilitate drug diffusion through the microconduits on the mucosa. Iontophoresis is then applied for a duration of 4 minutes.

Further advantageously, the swellable configuration allows for a further reduction in the time taken for anaesthetic onset. Using the hydrogel-forming swellable conductive MN can further increase the iontophoretic flux to further result in a reduction in the time taken for anaesthetic delivery, thereby achieving an easy, quick and painless drug delivery method which can be easily adopted by clinicians in clinical practice without the need for elaborate training.

Generally, in the conventional needle-syringe method, one cartridge containing 20 mg/ml of lidocaine is delivered per tooth for both adults and children. Depending on anatomical variations (e.g. thickness of alveolar bone) and local condition (e.g. presence of inflammation), more than one cartridge may be required, which means additional needle injections are administered to achieve the desired numbing effect. In the present approach, additional anaesthetic doses can similarly be administered if required, by applying more MN patches. However, a smaller dose of drug suffices to achieve anaesthesia compared to the conventional needle injection method. Hence, the present approach is an active delivery method driven by an electric current that is more efficient than passive diffusion.

### Example 5: Summary

Holistically, the present disclosure provides for a microneedle device for delivery of active agents to the skin or mucosa, said device comprises a base layer and a layer of microneedle structures, wherein the base layer is fabricated using a dissolvable polymer, and the layer of microneedle structures is fabricated onto on the base layer using a mixture comprising dissolvable polymer, conductive polymer, and active agents. In various embodiments, the dissolvable polymer may be hyaluronic acid (HA) and its derivatives (e.g. methacrylic hyaluronic acid). In various embodiments, the conductive polymer may be poly(3,4-ethylenedioxythiophene) polystyrene sulfonate (PEDOT:PSS). In various embodiments, the active agents may be anaesthetic agents.

The present disclosure also provides for use of the microneedle device as described above in transdermal delivery. In various embodiments, the device may be used for delivery of anaesthetic agents to the skin.

The present disclosure further provides for use of the microneedle device as described above in transmucosal delivery. In various embodiments, the device may be used for delivery of anaesthetic agents to sensory nerves in oral and/or maxillofacial surgery.

A method of transdermal delivery of active agents to the skin is provided herein, wherein the method comprises:

applying the microneedle device as described above on the skin;

optionally removing the base layer of the microneedle device;

if the active agents are cationic, applying the cathode of an iontophoresis device over the skin where the microneedle device was applied and the anode of the iontophoresis device onto another site of the skin next to the cathode; or

if the active agents are anionic, applying the anode of an iontophoresis device over the skin where the microneedle device was applied and the cathode of the iontophoresis device onto another site of the skin next to the anode; or

if the active agents are neutral, applying either anode or cathode of an iontophoresis device over the skin where the microneedle device was applied and the respective electrode onto another site of the skin next to the microneedle-applied area; and passing a voltage electrical current between the electrodes of the iontophoresis device.

There is also provided a method of transmucosal delivery of active agents to the oral mucosa, wherein the method comprises:

applying the microneedle device as described above on the mucosa;

optionally removing the base layer of the microneedle device;

if the active agents are cationic, applying the cathode of an iontophoresis device over the mucosa where the microneedle device was applied and applying the anode of the iontophoresis device onto the mucosa opposite the cathode; or

if the active agents are anionic, applying the anode of an iontophoresis device over the mucosa where the microneedle device was applied and the cathode of the iontophoresis device onto the mucosa opposite the anode; or

if the active agents are neutral, applying either anode or cathode of an iontophoresis device over the mucosa where the microneedle device was applied and the respective electrode onto the mucosa opposite the first-mentioned electrode; and passing a voltage electrical current between the electrodes of the iontophoresis device.

In various embodiments, the method comprises delivery of anaesthetic agents to sensory nerves in oral and/or maxillofacial surgery.

### Example 6: Commercial and Potential Applications

This present disclosure provides for a conductive MN array that can be combined with iontophoresis for efficient delivery of anaesthetics through the skin, mucosal layers, and/or bone. The anaesthetic delivery can be applied in, for example, dentistry.

As a non-limiting example, the present conductive MN array, together with iontophoresis, are suitable for use in dental applications that require a substance (e.g. drugs) to be delivered through the skin and/or mucosa and bone into the sensory nerves to achieve a particular effect. As demonstrated in the examples above, it can be used to deliver local anaesthetic (lidocaine) for anaesthetic purposes prior to a dental procedure. The present conductive MN array is potentially applicable to other parts of the body that involve the skin and/or mucosa and/or bone, be it for therapeutic or anaesthetic purposes.

The conductive property of the MN patch is one of the various features which allows the present MNs to perform two key functions. Firstly, the penetration of the present MNs into the oral mucosa creates micron-sized holes to allow the delivery of drug molecules. Secondly, the insertion of the present conductive MN patch into the oral mucosa tissue is able to alter the resistance of the skin/mucosal barrier. Such a feature of the MN array results in a synergistic effect when combined with iontophoresis. The development of a low resistance pathway within the oral mucosa allows for a stronger driving force enabling charged drug molecules to permeate quickly into the deep tissue layers. Specifically, the above examples already showed that both of the present MN arrays are capable of driving the anaesthetic drug into the bone tissue to result in quick drug onset. This is especially relevant and necessary for dental anaesthesia as the nerves supplying sensation to teeth are located deep within the bone.

The fabrication of the present conductive MN array can be achieved using simple micro molding techniques. In addition to the ease of manufacturing, the drug delivery system can also be extended to other transdermal drug delivery systems by changing the drug reservoir. Pre-existing iontophoresis machine which has been used as a medical device in the treatment of hyperhidrosis can be used without the need to develop new iontophoresis apparatus just for the present conductive MNs arrays. This development serves as an innovation that can reposition existing oral patches for other various uses.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A microneedle array comprising:
a base having microneedles disposed thereon, wherein each of the microneedles is formed of (i) a swellable and water-insoluble matrix comprising a crosslinked polymer or (ii) a water-soluble matrix comprising a water-soluble polymer; and
a conductive polymer incorporated in the swellable and water-insoluble matrix or the water-soluble matrix.

2. The microneedle array of claim 1, wherein the crosslinked polymer comprises an acrylate-crosslinked hydrophilic polymer, a furan-crosslinked hydrophilic polymer, or a catechol-crosslinked hydrophilic polymer, wherein the acrylate-crosslinked hydrophilic polymer comprises methacrylate-crosslinked hyaluronic acid, methacrylate-crosslinked polyvinyl alcohol, methacrylate-crosslinked poly(methylvinyl ether), or cross-linked poly(ethylene glycol) diacrylate, wherein the methacrylate-crosslinked hyaluronic acid is formed from hyaluronic acid having an average molecular weight ranging from 3 kDa to 300 kDa.

3. (canceled)

4. The microneedle array of claim 1, wherein the water-soluble polymer comprises hyaluronic acid, polyvinyl alcohol, poly(methylvinyl ether), poly(ethylene glycol), or poly(lactic-co-glycolic acid), wherein the hyaluronic acid has an average molecular weight ranging from 3 kDa to 300 kDa.

5. (canceled)

6. The microneedle array of claim 1, wherein the conductive polymer comprises 25 wt % or less of the swellable and water-insoluble matrix or the water-soluble matrix.

7. The microneedle array of claim 1, wherein the conductive polymer comprises poly(3,4-ethylenedioxythiophene)-poly(styrenesulfonate), polypyrrole, polyaniline, polythiophene, polyethyne, poly(p-phenylene), or poly(p-phenylene vinylene).

8. The microneedle array of claim 1, wherein each of the microneedles has a length of 1000 μm or less.

9. The microneedle array of claim 1, wherein the base comprises the crosslinked polymer or the water-soluble polymer which the swellable and water-insoluble matrix or the water-soluble matrix is respectively formed of.

10. The microneedle array of claim 1, wherein (i) the base has a surface for the active agent to be disposed thereon and/or (ii) the swellable and water-insoluble matrix or the water-soluble matrix further comprises the active agent disposed therein.

11. A device configured to deliver an active agent, the device comprising:
the microneedle array of claim 1;
and
an iontophoresis unit comprising an anode and a cathode connectable to the microneedle array, wherein the iontophoresis unit is operable to deliver the active agent from the microneedle array.

12-20. (canceled)

21. The device of claim 11, wherein the active agent comprises an anaesthetic agent and/or a drug.

22. A method of producing the microneedle array of claim 1,
wherein the method comprises:
providing an aqueous solution in a mold, wherein the aqueous solution comprises (i) a functionalized polymer, the conductive polymer and a photoinitiator, or (ii) the water-soluble polymer and the conductive polymer;
irradiating the aqueous solution to form the microneedle array when the aqueous solution comprises the functionalized polymer, the conductive polymer and the photoinitiator; and
removing the microneedle array from the mold.

23-24. (canceled)

25. The method of claim 22, wherein the functionalized polymer comprises an acrylate-functionalized hydrophilic polymer, a furan-functionalized hydrophilic polymer, or a catechol-functionalized hydrophilic polymer, wherein the acrylate-functionalized hydrophilic polymer comprises methacrylate-functionalized hyaluronic acid, methacrylate-functionalized polyvinyl alcohol, methacrylate-functionalized poly(methylvinyl ether), or diacrylate-functionalized poly(ethylene glycol).

26. (canceled)

27. The method of claim 22, wherein providing the aqueous solution comprises (i) mixing the functionalized polymer with the conductive polymer and a photoinitiator or (ii) mixing the water-soluble polymer with the conductive polymer.

28-29. (canceled)

30. The method of claim 22, wherein the mold comprises a plurality of cavities shaped to form the microneedles, wherein each of the plurality of cavities has a depth of 1000 μm or less.

31. (canceled)

32. The method of claim 22, further comprising centrifuging the mold with the aqueous solution provided therein.

33. A method of delivering an active agent to a subject through the device of claim 11, the method comprising:
applying the microneedle array on the subject;
placing the anode and the cathode on the subject; and
operating the iontophoresis unit to deliver the active agent from the microneedle array.

34. The method of claim 33, wherein applying the microneedle array comprises inserting the microneedles into a first surface of the subject.

35. The method of claim 34, wherein placing the anode and the cathode on the subject comprises:
(i) arranging the anode on the first surface proximal to where the microneedle is applied and arranging the cathode on a second surface distal to where the anode is arranged when the active agent is anionic; or
(ii) arranging the cathode on the first surface proximal to where the microneedle is applied and arranging the cathode on a second surface distal to where the cathode is arranged when the active agent is cationic; or
(iii) arranging either the anode or the cathode on the first surface proximal to where the microneedle is applied and arranging the cathode or the anode, respectively, on a second surface to where the anode or cathode is arranged, respectively, when the active agent is neutral.

36. The method of claim 33, wherein operating the iontophoresis unit comprises passing an electrical current between the anode and the cathode to establish a voltage for delivering the active agent from the microneedle array.

37. The method of claim 33, wherein delivering the active agent from the microneedle array comprises delivering the active agent to and/or through
(i) a dermal layer of the subject; and/or
(ii) a mucosa of the subject; and/or
(iii) a deep dermis layer of the subject; and/or
(iv) a bone of the subject.
